(19) United States
(12) Patent Application Publication
(10) Pub. No.: US 2008/0113101 A1
(43) Pub. Date: May 15, 2008

(54) ORGANIC-ELECTROLUMINESCENCE-MATERIAL-CONTAINING SOLUTION, METHOD FOR FORMING THIN FILM OF ORGANIC ELECTROLUMINESCENCE MATERIAL, THIN FILM OF ORGANIC ELECTROLUMINESCENCE MATERIAL AND ORGANIC ELECTROLUMINESCENCE DEVICE

(75) Inventors: Tetsuya Inoue, Sodegaura-shi (JP); Masakazu Funahashi, Sodegaura-shi (JP); Mineyuki Kubota, Sodegaura-shi (JP); Mitsunori Ito, Sodegaura-shi (JP); Chishio Hosokawa, Sodegaura-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Idemitsu Kosan Co., Ltd., Chiyoda-ku (JP)

(21) Appl. No.: 11/564,058

(22) Filed: Nov. 28, 2006

(30) Foreign Application Priority Data
Nov. 9, 2006 (JP) 2006-304627

(51) Int. Cl. C09K 11/06; B05D 3/00

(52) U.S. Cl. 427/384; 252/301.16

(57) ABSTRACT

An organic EL material-containing solution contains an organic EL material, a solvent and a viscosity control agent. The organic EL material contains a host and a dopant.

The host is a compound shown by Formula (1) below and has a solubility of 2 wt % or higher in the solvent. The solvent is an aromatic solvent, while the viscosity control agent is an alcohol type solution or an alkyl-substituted aromatic solution having 4 or more carbon atoms.

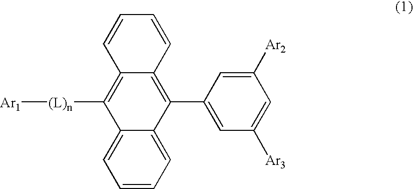

### BACKGROUND OF THE INVENTION

1. Field of the Invention

The present invention relates to an organic EL material-containing solution, a method for forming a thin film of an organic EL material, the thin film of the organic EL material and an organic EL device. More specifically, the present invention relates to an organic EL material-containing solution which is used in forming an organic thin film that forms the organic EL device by a coating method.

2. Description of Related Art

There has been known an organic EL (Electroluminescence) device that utilizes luminescence of an organic compound.

The organic EL device includes a plurality of organic thin films laminated between an anode and a cathode.

As organic EL materials, high-molecular materials and low-molecular materials are known. In view of simple synthetic pathway and capability of high degree of purification, low-molecular organic EL materials have been developed. Among those developed low-molecular organic EL materials, organic EL materials having excellent efficiency, lifecycle and color purity have been proposed and put to practical use.

Vacuum deposition is employed for forming thin films using the low-molecular EL organic material, where the low-molecular EL organic material is sublimated with good thermal stability to vapor-deposit the thin films on a substrate, thereby obtaining a high-quality organic EL device (see Document1: WO2004/018587).

However, the vapor deposition requires equipment capable of producing high vacuum and complicated manufacturing process.

Meanwhile, a coating method has been known as a film formation method for the organic EL materials.

The coating method is typically used in film forming using the high-molecular organic EL material, in which an organic EL material solved in a solvent to form thin films of the organic EL material. The coating method has an advantage in which the thin films of the organic EL material can be easily formed. In order to form the thin films of the organic EL material by the coating method, the organic EL material needs to be solved in a solvent, and a coating composition that is prepared by solving a high-molecular organic El material in a solvent has been typically known.

Examples of the solvent include toluene, xylene, tetralin, mesitylen, cyclohexylbenzene and isopropylbiphenyl (see, Document 2: WO2005/059267, Document 3: JP-A-2002-313561 and Document 4: JP-A-2004-119351).

When thin films are formed from the low-molecular organic EL materials by the coating method, there may arise a problem in dissolving an arbitrary low-molecular organic EL material in the solvents above because the low-molecular organic materials have poor solubility.

The coating method is applicable to materials having solubility of a predetermined value or higher (e.g., 0.5 wt % or higher). However, the solubility of the low-molecular EL organic materials is generally in the range from 0.1 wt % to 0.2 wt %, and the coating method has not been applicable to the low-molecular organic EL materials due to the low solubility.

Although the coating method has been recently found out to be applicable to the film formation of the low-molecular materials recently (see, Document 5: JP-A-2006-190759), the solubility is still insufficient. In addition, performance (luminescence efficiency and lifecycle) of a resulting organic EL device is not good enough.

Meanwhile, when the low-molecular organic EL material is dissolved in a solvent, process suitability may be low due to its low solution viscosity.

Inkjet printing and nozzle printing are known as the coating methods for the film formation, where the viscosity needs to be 1 cP or higher in the nozzle printing and 1.5 cP or higher in the ink jet printing.

When the high-molecular organic EL material is employed, the solution viscosity can be high only by dissolving the high-molecular organic EL material in the solvent.

On the other hand, when the low-molecular organic EL material is employed, the solution viscosity cannot be high only by dissolving the low-molecular organic EL material in the solvent. For example, even when the low-molecular organic EL material is dissolved in the solvents such as toluene and the xylene, the solution viscosity is still lower than 1 cP. Accordingly, a thickening component for increasing the viscosity has to be added.

Although an alcohol type solution is typically known as the thickening component, the alcohol type solution is a poor solvent for the low-molecular organic EL material.

Addition of the poor solvent as the thickening component as described above causes the solubility to become even lower.

In addition, it has been recognized, as another problem in employing the low-molecular organic EL material, that a solid component is precipitated over time.

Even if the solubility or the viscosity is simply adjusted, the solid component is precipitated over time, which causes cluster when the films are formed by the coating method, thus resulting in unevenness of the films. In addition, when the films are formed by the ink jet printing, head nozzles will be clogged.

Under such circumstance, a pot life is extremely short and a user has to use the solution quite shortly after the solution is prepared, which leaves a problem in the process suitability.

Although Document 6 (JP-A-2005-259523) discloses an ink using a mixed solvent of a good solvent and a poor solvent, the ink is not good enough for practical use due to the disadvantage described above.

With the problems described above, the films cannot be formed easily and at low cost from the low-molecular organic EL material that has excellent luminescence efficiency, lifecycle and color purity using the coating method, which is a severe obstacle in full-scale practical application of the organic EL materials.

### SUMMARY OF THE INVENTION

An object of the present invention is to provide an organic EL material-containing solution that is free from the problems described above and therefore can be applied to a coating method.

Another object of the present invention is to provide a method for forming a thin film of an organic EL material, the thin film of the organic EL material and an organic EL device.

An organic EL material-containing solution according to an aspect of the present invention contains an organic EL material, a solvent and a viscosity control agent, in which the organic EL material contains a host and a dopant, the host is a compound shown by Formula (1) below, and the host has a solubility of 2 wt % or higher in the solvent,

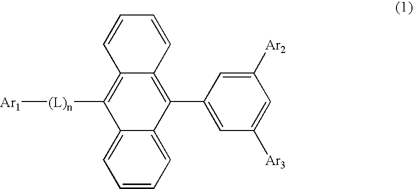

(Ar₁ to Ar₃ each representing a substituted or unsubstituted aryl group or heteroaryl group having 5 to 50 nuclear atoms or a condensed aromatic group having 10 to 30 carbon atoms, L representing a single bond or a divalent linking group, the divalent linking group being a substituted or unsubstituted arylene group or heteroarylene group having 5 to 50 nuclear atoms, n representing an integer of 1 to 4).

As shown by Formula (1), by attaching a substituent group at a meta position to a phenyl group bonded to a central anthracene skeleton, solubility in a solvent can be enhanced. Such material exhibits high performance as the organic EL material. Accordingly, the organic EL material-containing solution suitable for film formation by the coating method can be obtained.

In addition, such compound exhibits high performance as an organic EL material.

The substituent group is attached at the position 9, 10 of the central anthracene skeleton in the aspect of the present invention. However, in the related art, the substituent group is attached at positions 1 to 4 and 5 to 8 to obtain solubility. Therefore, the performance as the organic EL material is not good enough with insufficient luminescence performance and lifecycle.

In contrast, the compound according to the aspect of the present invention exhibits high solubility in the solvent, and further, the performance as the organic EL material is high.

In addition, since the solubility of the host material can be made sufficiently high, a viscosity control agent for viscosity control required in the coating process can be added. Such viscosity control agent is typically a poor solvent. However, since the solubility of the host is sufficiently high, even the viscosity control agent as the poor solvent can be added while securing a required amount of dissolution Therefore, the organic EL material-containing solution suitable for film formation by the coating method can be obtained.

A description about the host material and the dopant material will be given.

An organic EL device is formed by lamination of layers including a hole injection layer/a hole transport layer/a luminescent layer/an electron transport layer/an electron injection layer and the like. The luminescent layer is formed from the host material and the dopant material, where an energy transmission or the like is generated from the host material to the dopant material, so that the dopant material shows a luminescent function.

The dopant material is added (doped) to the host material by, for instance, and the ratio of the dopant material/the host material is 0.01 to 20 wt %. The host material forms a major part (e.g., 80% or more) of the luminescent layer having a thickness of, for instance, 30 nm to 100 nm. With the arrangement, in order to form the luminescent layer by the coating process, a predetermined amount of the host material has to be dissolved in the organic EL material-containing solution.

In this regard, according to the aspect of the present invention, the organic EL material-containing solution suitable for film formation by the coating method can be obtained.

Note that L represents a single bond or a substituted or unsubstituted arylene group or heteroarylene group having 5 to 50 nuclear atoms as a divalent linking group, and preferably a condensed aromatic group having 10 to 30 carbon atoms.

According to aspect of the present invention, it is preferable that Ar₁ to Ar₃ each are a substituted or unsubstituted aryl group having 5 to 50 nuclear atoms.

According to aspect of the present invention, it is preferable that Ar₁ to Ar₃ each are a substituted or unsubstituted phenyl group or a naphthyl group.

By providing the phenyl group or the naphthyl group as Ar₁ to Ar₃, both the performance as the host material and the lifecycle can be enhanced. Accordingly, the host material that is excellent in both the solubility and the performance as the EL material can be obtained.

According to aspect of the present invention, it is preferable that the host is a compound shown by Formula (2) below instead of the compound shown above in Formula (1),

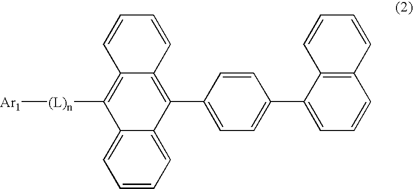

(Ar₁ representing a substituted or unsubstituted aryl group or heteroaryl group having 5 to 50 nuclear atoms, L representing a single bond or a divalent linking group, the divalent linking group being a substituted or unsubstituted arylene group or heteroarylene group having 5 to 50 nuclear atoms or a condensed aromatic group having 10 to 30 carbon atoms, n representing an integer of 1 to 4).

As shown by Formula (2), by bonding the naphthyl group in the para position to the central anthracene skeleton with the phenyl group interposed therebetween, the solubility can be enhanced.

In addition, such compound exhibits high performance as the organic EL material.

Therefore, the organic EL material-containing solution suitable for the film formation by the coating method can be obtained.

According to the aspect of the present invention, it is preferable that Ar₁ is a substituted or unsubstituted aryl group having 5 to 50 nuclear atoms.

According to the aspect of the present invention, it is preferable that Ar₁ is a substituted or unsubstituted aryl group having 5 to 50 nuclear atoms.

According to the aspect of the present invention, it is preferable that Ar₁ is a substituted or unsubstituted phenyl group or a naphthyl group.

The right part of the above formula contributes to improvement of the solubility, while the left part of the above formula contributes to improvement of the performance as the organic EL material by selecting a suitable substituent group. For example, by selecting the phenyl group or the naphthyl group for the substituent group, both of performance as the host material and the lifecycle can be enhanced.

Accordingly, the host material that is excellent in both the solubility and the performance as the EL material can be obtained.

According to the aspect of the present invention, it is preferable that n is 1 or 2.

When n is too large, the performance as the organic EL material cannot be sufficiently exhibited. Accordingly, by setting n to 1 or 2, a material excellent in the luminescence property and lifecycle can be obtained. Such material also has a high solubility, thus providing the organic EL material-containing solution that is suitable for the film forming by the coating method.

According to the aspect of the present invention, it is preferable that the dopant is a styrylamine derivative shown by Formula (3) below, the dopant having a substituent group that is a straight-chained or branched alkyl group having 2 to 6 carbon atoms or a cycloalkyl group having 5 to 10 carbon atoms, and the dopant material has a solubility of 0.5 wt % or higher in the solvent,

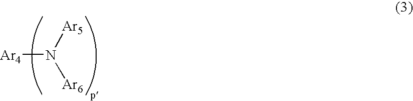

(at least one of Ar₄ to Ar₆ containing a styryl group, preferably, Ar₄ being a group selected from the group containing a phenyl group, biphenyl group, a terphenyl group, a stilbene group and a distyrylaryl group, Ar₅ and Ar₆ each representing a hydrogen atom or an aromatic group having 6 to 20 carbon atoms, p′ representing an integer of 1 to 4)

In the formula above, the aromatic group having 6 to 20 carbon atoms is preferably a phenyl group, a naphthyl group, an anthracenyl group, a phenanthryl group, a terphenyl group or the like.

According the aspect of the present invention, it is preferable that the dopant 10 material is, instead of the styrylamine derivative shown by Formula (3), a substituted derivative of an arylamine shown by Formula (4) below, the substituted derivative of the arylamine having as a substituent group a straight-chained or branched alkyl group having 2 to 6 carbon atoms or a cycloalkyl group having 5 to 10 carbon atoms,

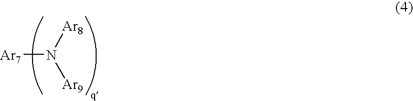

### (Ar₇ to Ar₉ each representing a substituted or unsubstituted aryl group having 5 to 40 nuclear carbon atoms, q′ representing an integer of 1 to 4).

In the formula above, the aryl group having the 5 to 40 nuclear atoms is preferably phenyl, naphthyl, anthracenyl, phenanthryl, pyrenyl, chrysenyl, coronyl, biphenyl, terphenyl, pyrrolyl, furanyl, thiophenyl, benzothiophenyl, oxadiazolyl, diphenyl anthracenyl, indolyl, carbazolyl, pyridyl, benzoquinolyl, fluorenyl, fluoranthenyl, acenaphthofluoranthenyl, stilbene, a group represented by General Formula (A) or (B) below or the like.

In General Formula (A), r represents an integer of 1 to 3.

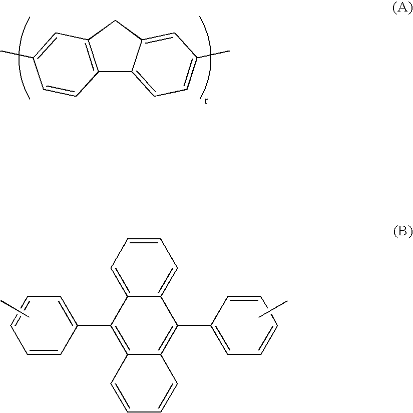

The aryl group having 5 to 40 nuclear atoms may be substituted by a substituent group, in which the substituent group is preferably an alkyl group having 2 to 6 carbon atoms (e.g., an ethyl group, a methyl group, an isopropyl group, an n-propyl group, an s-butyl group, a t-butyl group, a pentyl group, a hexyl group, a cyclopentyl group and a cyclohexyl group).

In the solution having such composition, the low-molecular organic EL material cannot exhibit sufficient solubility in the solvent. However, by providing the straight-chained or branched alkyl group having 2 to 6 carbon atoms or the cycloalkyl group having 5 to 10 carbon atoms as the substituent group, the solubility of the dopant becomes equal to or higher than the predetermined value and therefore the solubility of the low-molecular organic EL material can be enhanced.

Since the material having a sufficient solubility as described above is used as a solute, the solution can be prepared by dissolving the material as the solute and further adding thereto the viscosity control agent as a thickener for viscosity control.

Accordingly, the organic EL material-containing solution having a viscosity of 1 cp or higher and an amount of dissolution of 0.5 wt % or higher can be obtained.

Since the low-molecular organic EL materials typically have poor solubility and do not exhibit high viscosity even when being dissolved, it is difficult to select a solvent that can dissolve the low-molecular organic EL materials while achieving a sufficient viscosity.

However, by separately selecting a solvent for dissolving the low-molecular organic EL material and a viscosity control agent for controlling the viscosity, both the sufficient solubility and viscosity can be achieved at the same time.

Although the low-molecular organic EL material typically has poor solubility, it is insufficient simply to select a material that can be dissolved in a solvent to a certain extent required for coating.

Unlike the high-molecular EL material, since the low-molecular organic EL material does not exhibit sufficient viscosity as a solution, a thickening component requires to be added.

As the thickening component, the viscosity control agent that serves as a thickener is added as an additive. However, the viscosity control agent is typically a poor solvent for the low-molecular organic EL material.

Since, the solution requires to have a sufficient solubility for the coating even with the viscosity control agent being added to achieve a sufficient viscosity, the amount of dissolution of the low-molecular organic EL material in the solvent requires to be a value higher than a solubility required as a coating solution.

With this regard, according to the aspect of the present invention, compounds that has a solubility of a predetermined value or higher are selected from compounds soluble in the solvent based on experiments. In short, by selecting specific compounds, the solubility equal to or larger than a predetermined value is achieved. Accordingly, the organic EL material-containing solution that contains the low-molecular material in a uniformly dissolved state can be obtained even when the viscosity control is sufficiently performed, which is preferable for the coating.

Further, even when the low-molecular organic EL material can be dissolved in the solvent, the material is precipitated in a relatively short time (e.g., in few hours to few days). The high-molecular organic EL material is not typically precipitated once it is dissolved in the solvent. Precipitation is another problem in using the low-molecular organic EL material of the coating.

According to the aspect of the present invention, an experiment has been performed to check generation of precipitate as time advances after dissolution in order to select materials that can be dissolved in the solvent by a predetermined amount or more and that do not precipitate for a predetermined time period or longer, the materials being a host material having a specific structure and a dopant material having a specific substituent group.

Consequently, the pot life of the organic EL material-containing solution becomes sufficiently long, which contributes to the practical application of the organic EL material-containing solution.

Meanwhile, “having the branched alkyl group having 2 to 6 carbon atoms or the cycloalkyl group having 5 to 10 carbon atoms as the substituent group” means that such substituent group is contained at an end of the molecular structure. In other words, one of the Ar₄ to Ar₉ attached at the end of the molecule has the substituent group described above.

By selecting the solvent from an aromatic solvent, a halogen type solvent and an ether type solvent, the low-molecular organic EL material can be dissolved in the solvent by a required amount or more (e.g., 2 wt % or higher).

Further, by selecting the viscosity control agent from an alcohol type solution, a ketone type solution and a paraffin type solution, the viscosity can be increased and controlled so as to be suitable for various types of coating such as ink jet printing, nozzle printing and spin coating.

Note that the solvent is at least one type selected from the group consisting of the aromatic solvent, the halogen type solvent and the ether type solvent, and two or more of them may be used in combination.

Similarly, the viscosity control agent is at least one type selected from the group consisting of the alcohol type solution, the ketone type solution, the paraffin type solution, the alkyl-substituted aromatic solution and the alkyl-substituted aromatic solution having 4 or more carbon atoms, and two or more of them may be used in combination.

The “alkyl-substituted aromatic solution having 4 or more carbon atoms” is an alkyl-substituted group that is aromatic and has 4 or more carbon atoms. Although the upper limit of the number of carbon atoms of the alkyl-substituted group is not particularly set, the upper limit may be, for instance, approximately 500.

According to the aspect of the present invention, it is preferable that the solvent is the aromatic solvent, and the viscosity control agent is the alcohol type solution or the alkyl-substituted aromatic solution having 4 or more carbon atoms.

Here, when the alcohol type solution is selected as the viscosity control agent, storage of the resulting solution requires close attention since the alcohol type solution easily absorbs moisture. However, by selecting the alkyl-substituted aromatic solution having 4 or more carbon atoms as the viscosity control agent, which is hydrophobic, the storage of the resulting solution can be facilitated.

In addition, the alkyl-substituted aromatic solution having 4 or more carbon atoms is capable of controlling the viscosity by changing the structure of the alkyl group (for instance, by prolonging an alkyl chain).

On the other hand, the alcohol type solution is preferable in preparing a solution that is suitable for a film forming process requiring high solution viscosity (e.g., ink jet printing) due to its high viscosity.

The alcohol type solution is suitable for controlling coating process suitability also in terms of its high boiling point.

A type or an adding amount of the viscosity control agent can be properly selected in accordance with the viscosity required for various types of film forming processes.

An organic EL material-film forming method according to another aspect of the present invention includes: a dropping step for dropping the organic EL material-containing solution in a film formation area; and a film forming step for evaporating the solvent in the organic EL material-containing solution dropped in the dropping step to form a thin film of the organic EL material.

A thin film of an organic EL material according to still another aspect of the present invention is formed by the organic EL material-film forming method described above.

An organic EL device according to yet another aspect of the invention includes the thin film of the organic EL material described above.

It should be noted that the organic EL material-containing solution of the present invention may be used by adding additives thereto such that the viscosity, boiling point and concentration are controlled to be suitable for a certain coating method, in addition to be used as it is as the solution for the coating.

### DETAILED DESCRIPTION OF PREFERRED EMBODIMENT(S)

The present invention will be described below in detail.

An organic EL material-containing solution of the present invention is prepared by dissolving an organic EL material in a solvent.

The organic EL material-containing solution contains a host and a dopant.

The host may be exemplified by anthracene compounds shown below.

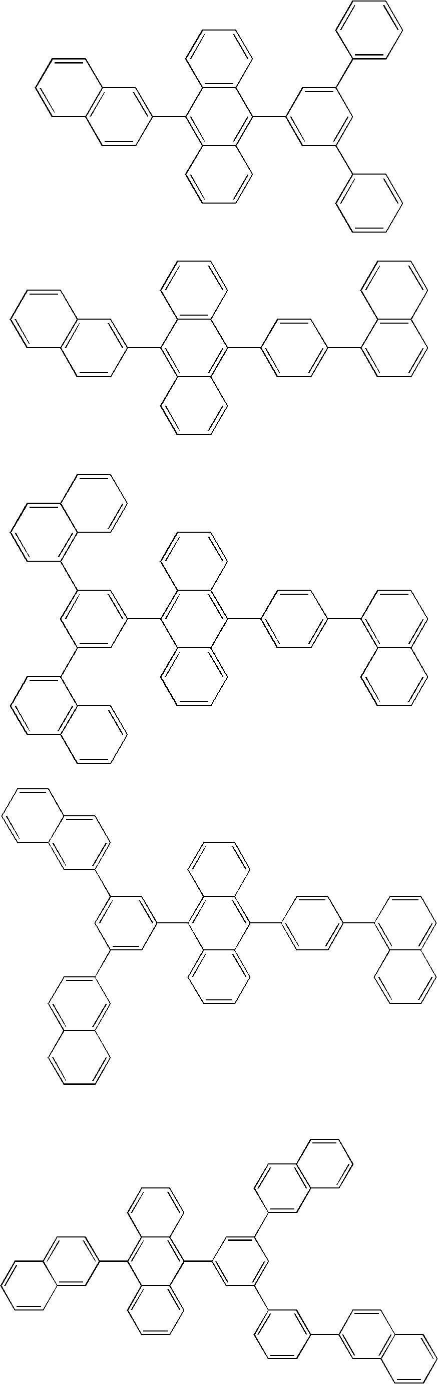

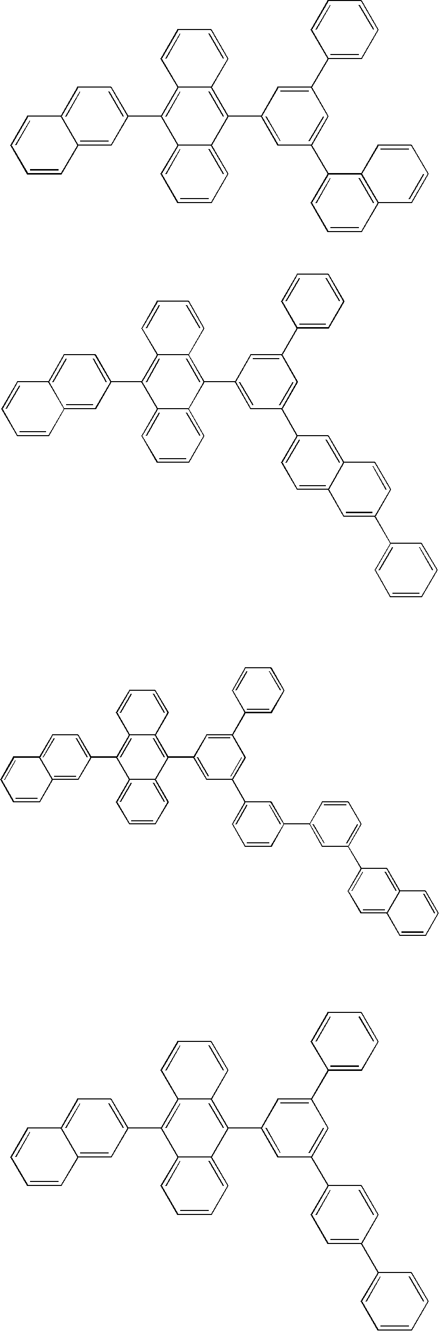

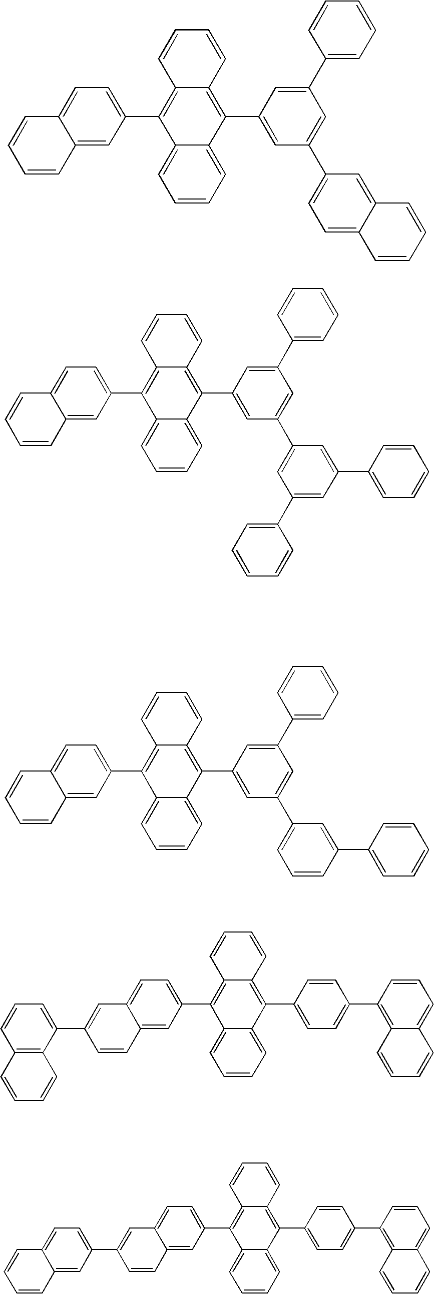

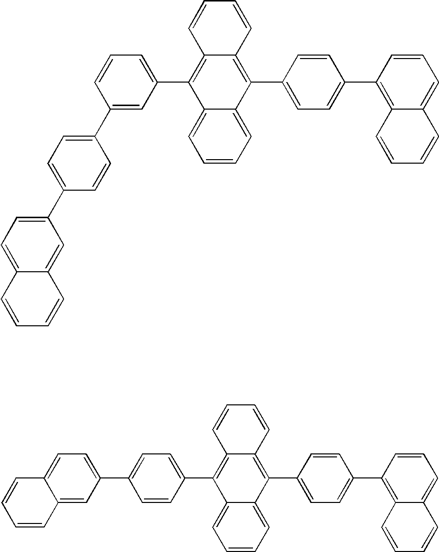

The dopant material may be exemplified by condensed aromatic amines or styrylamines shown below.

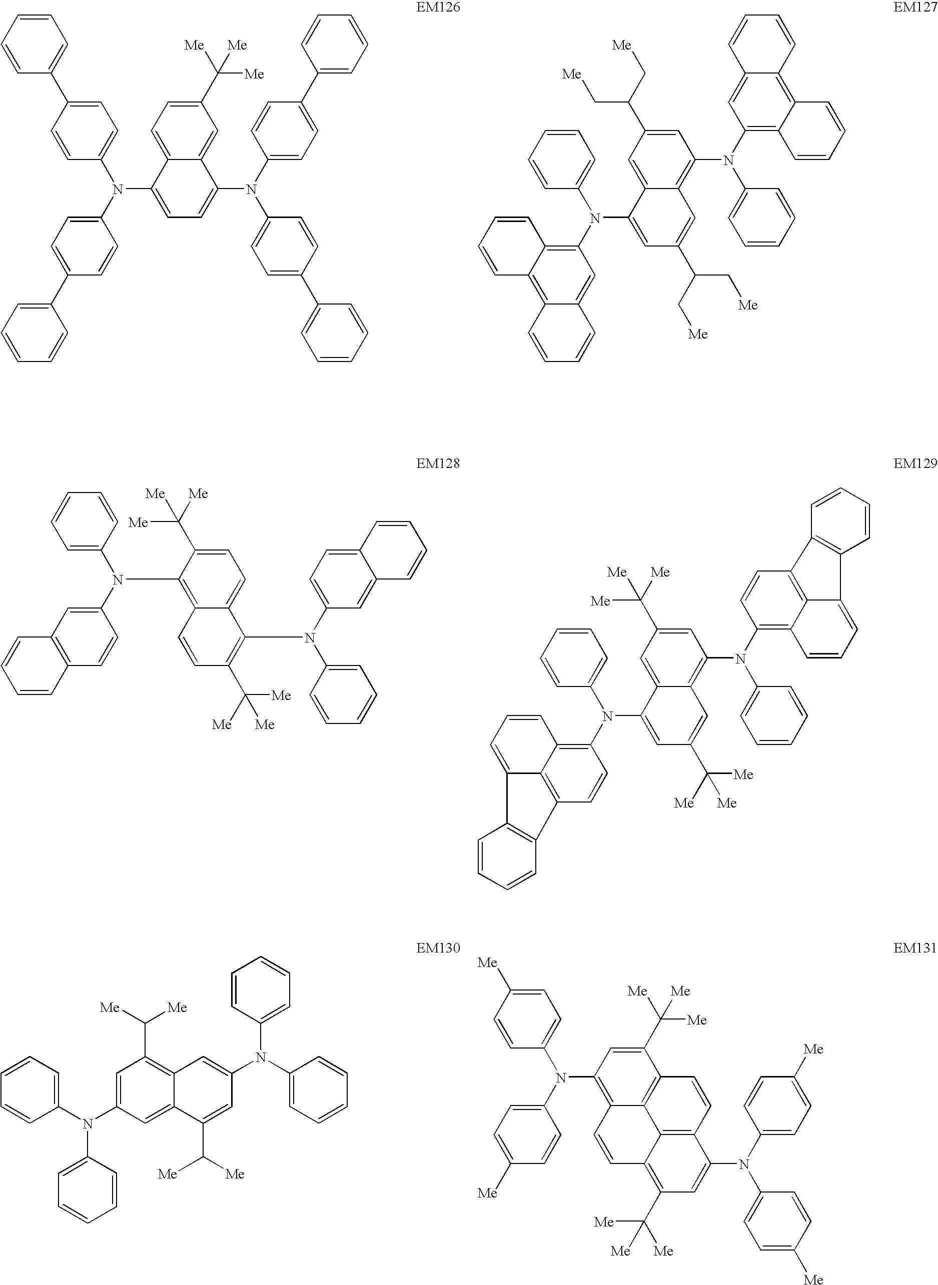

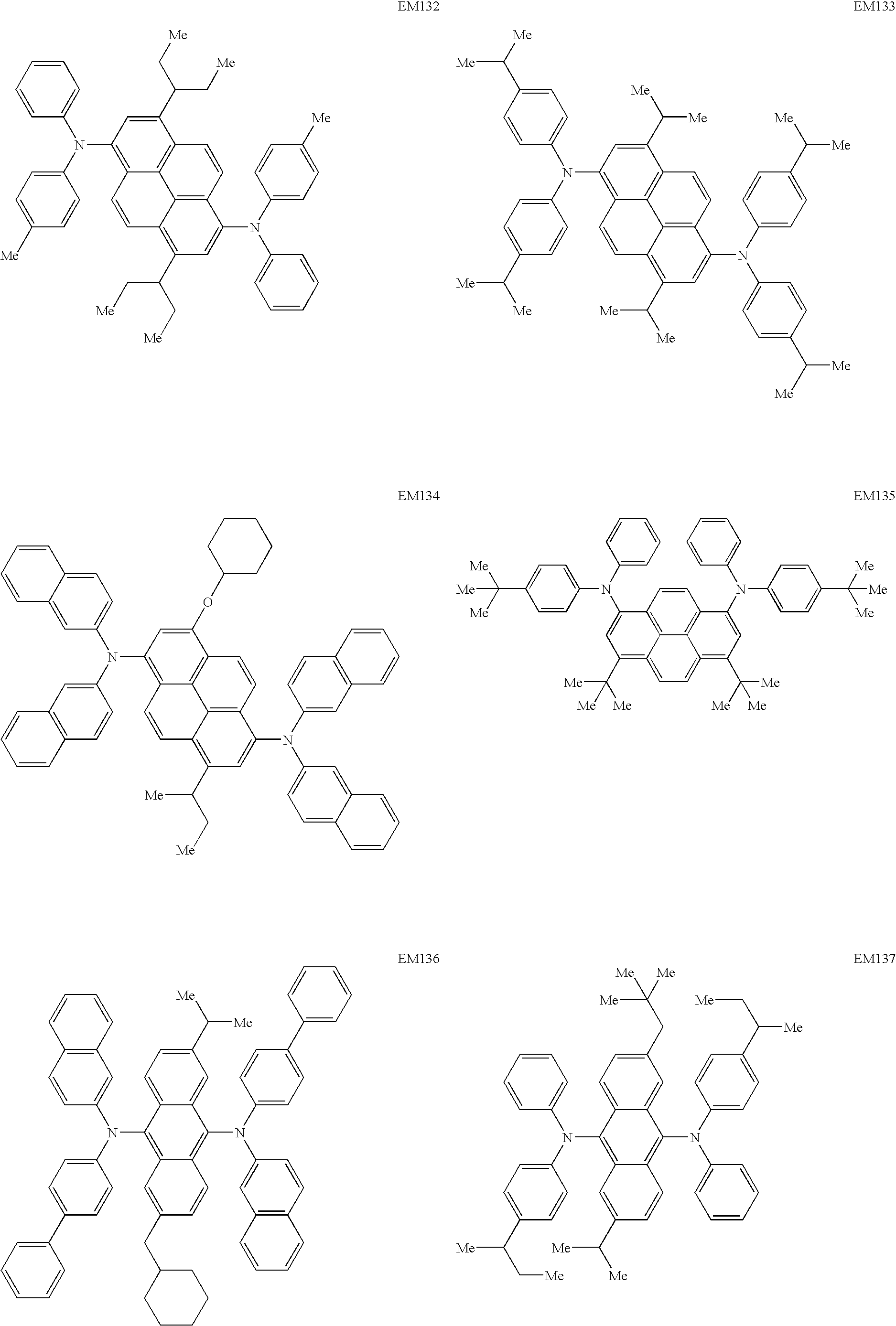

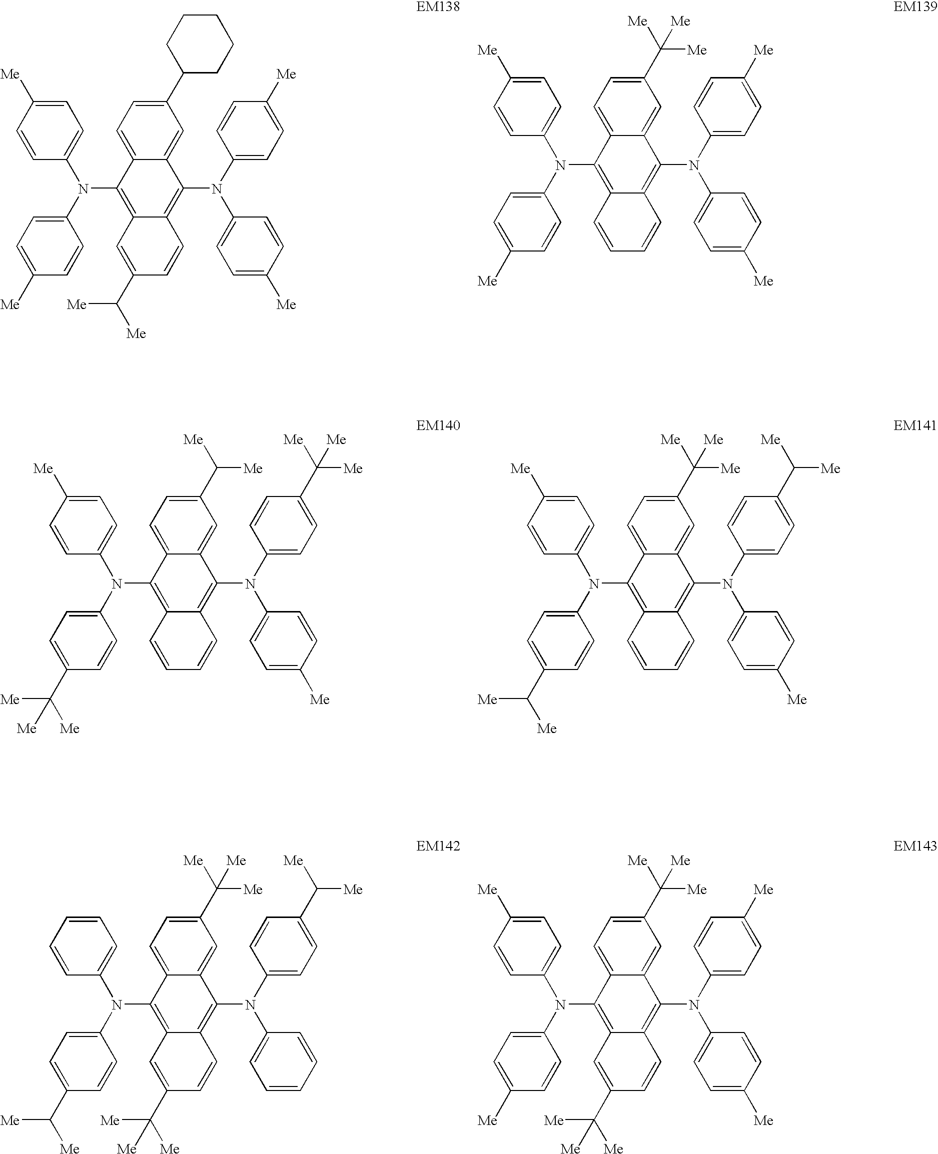

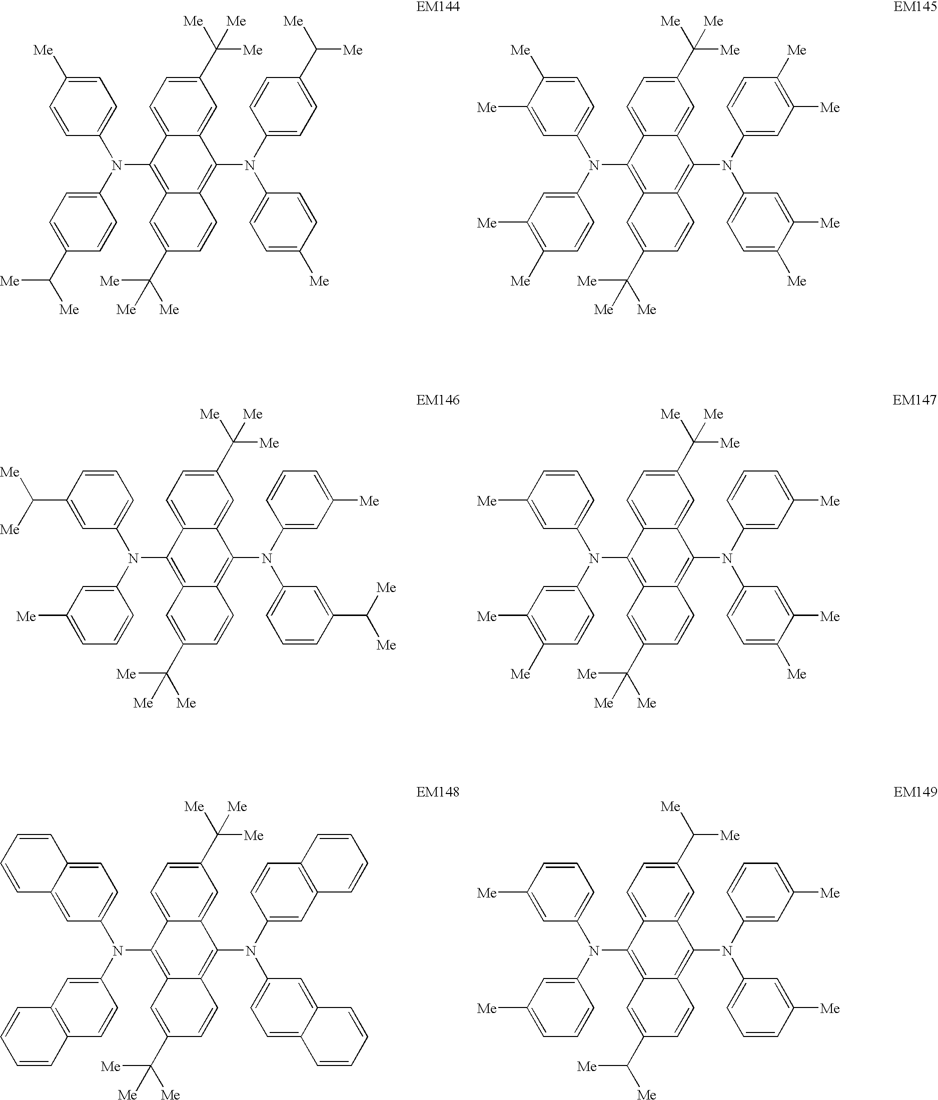

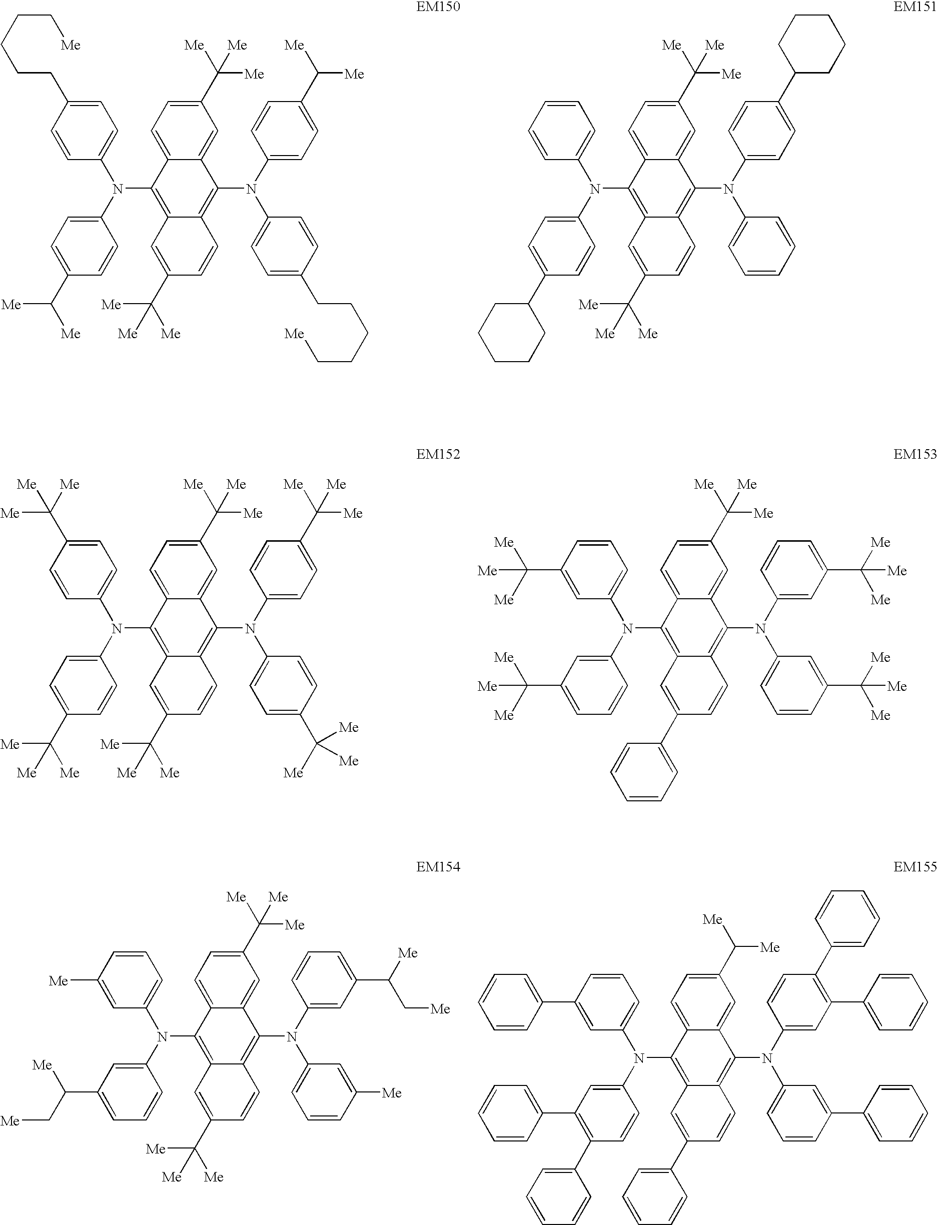

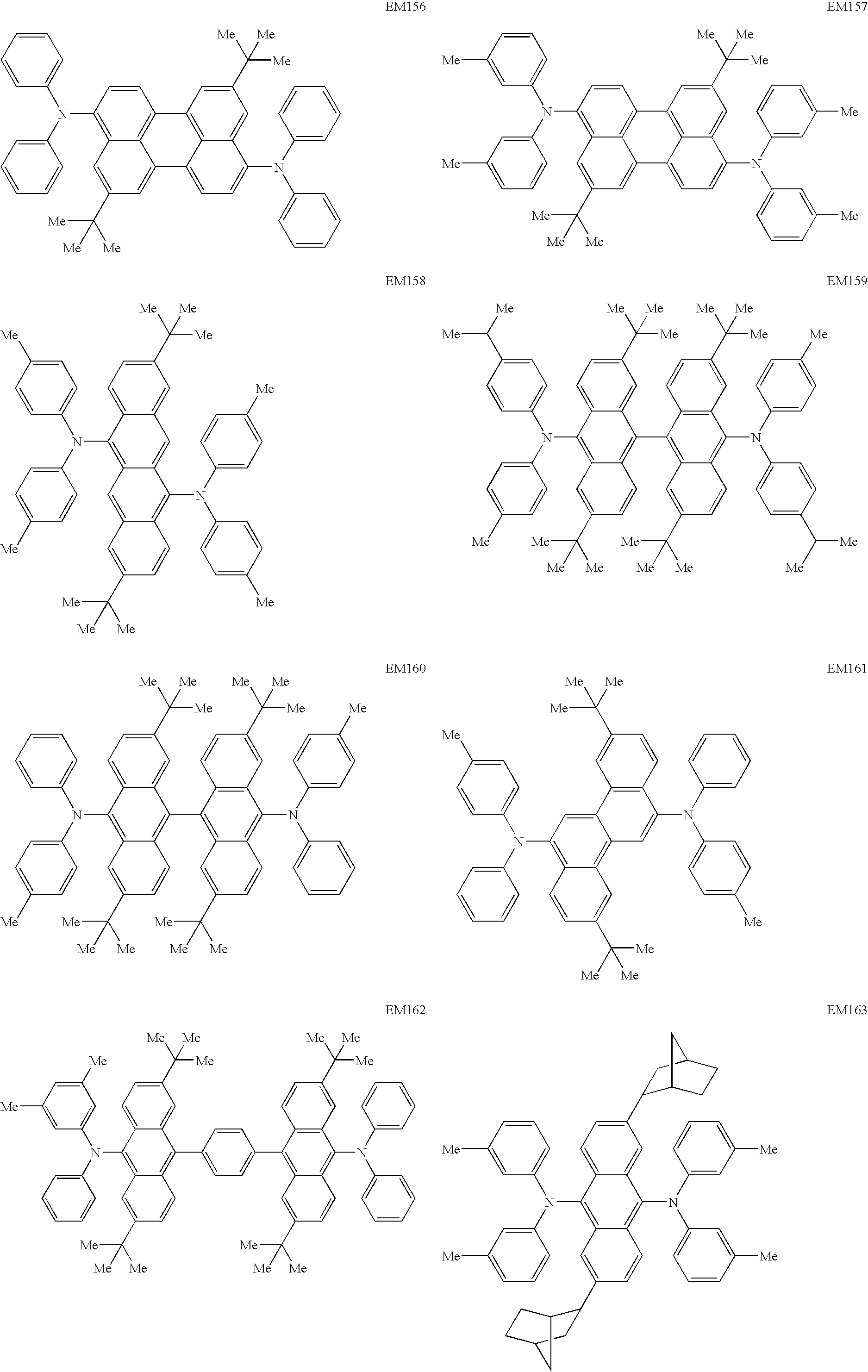

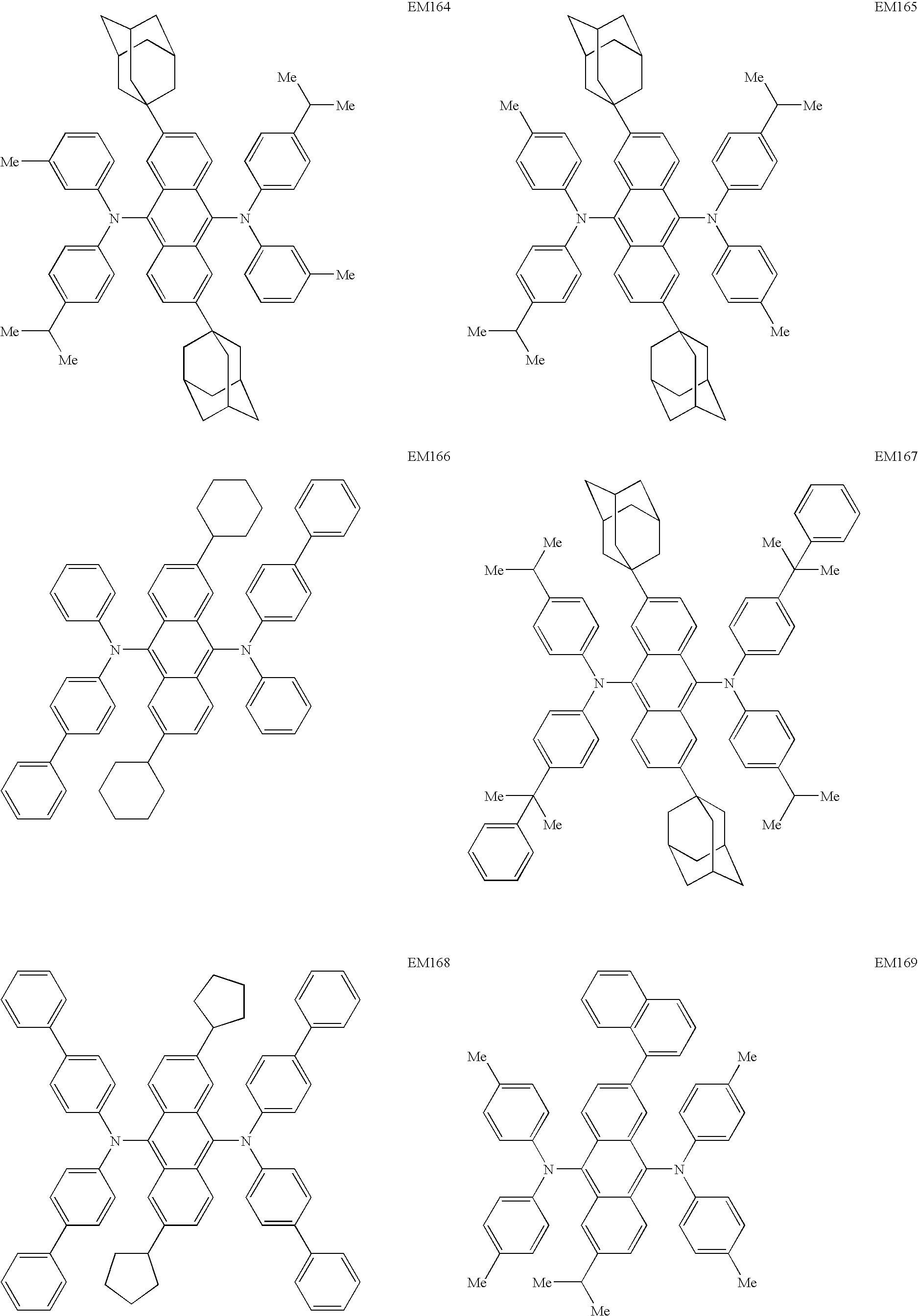

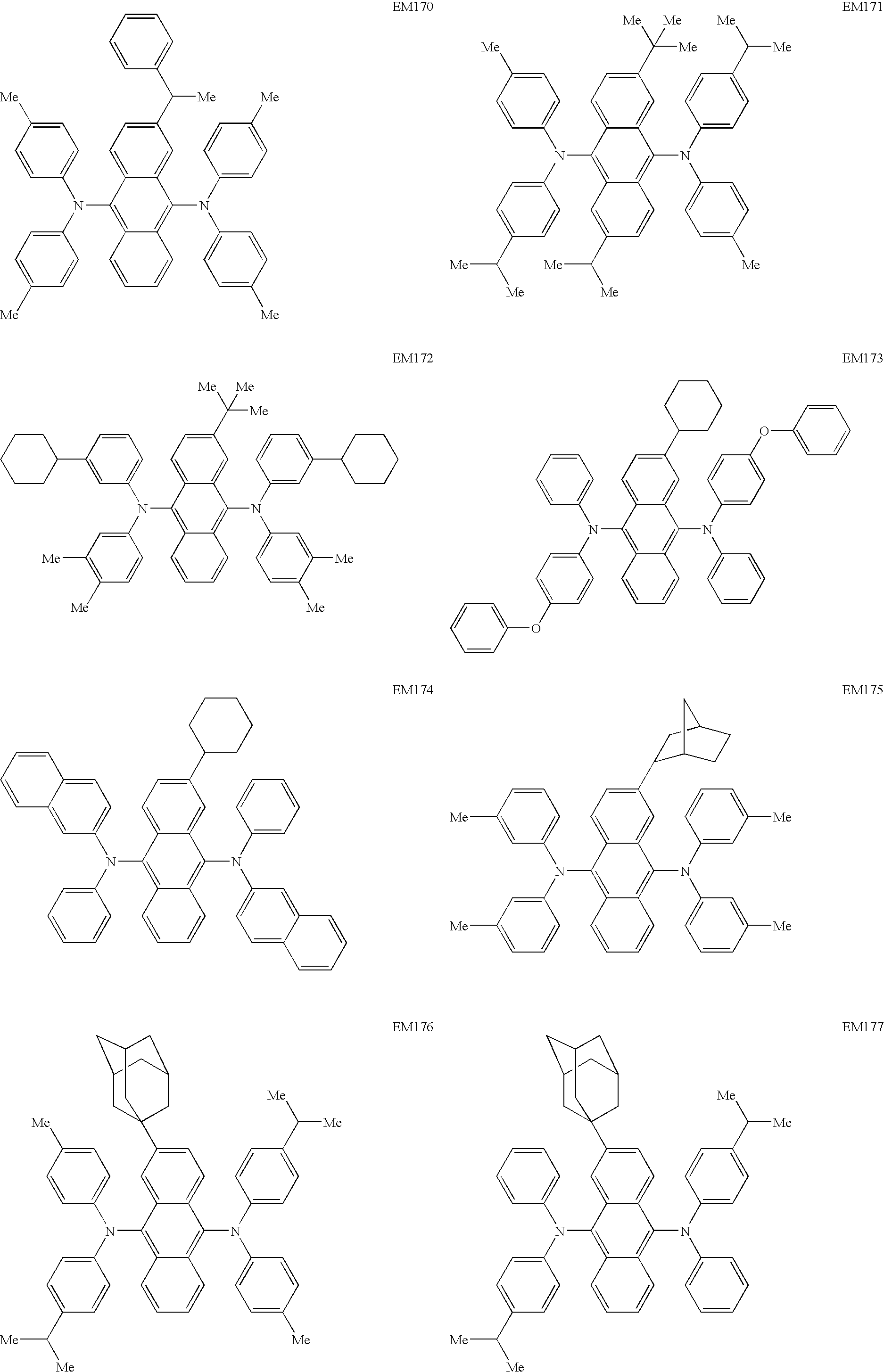

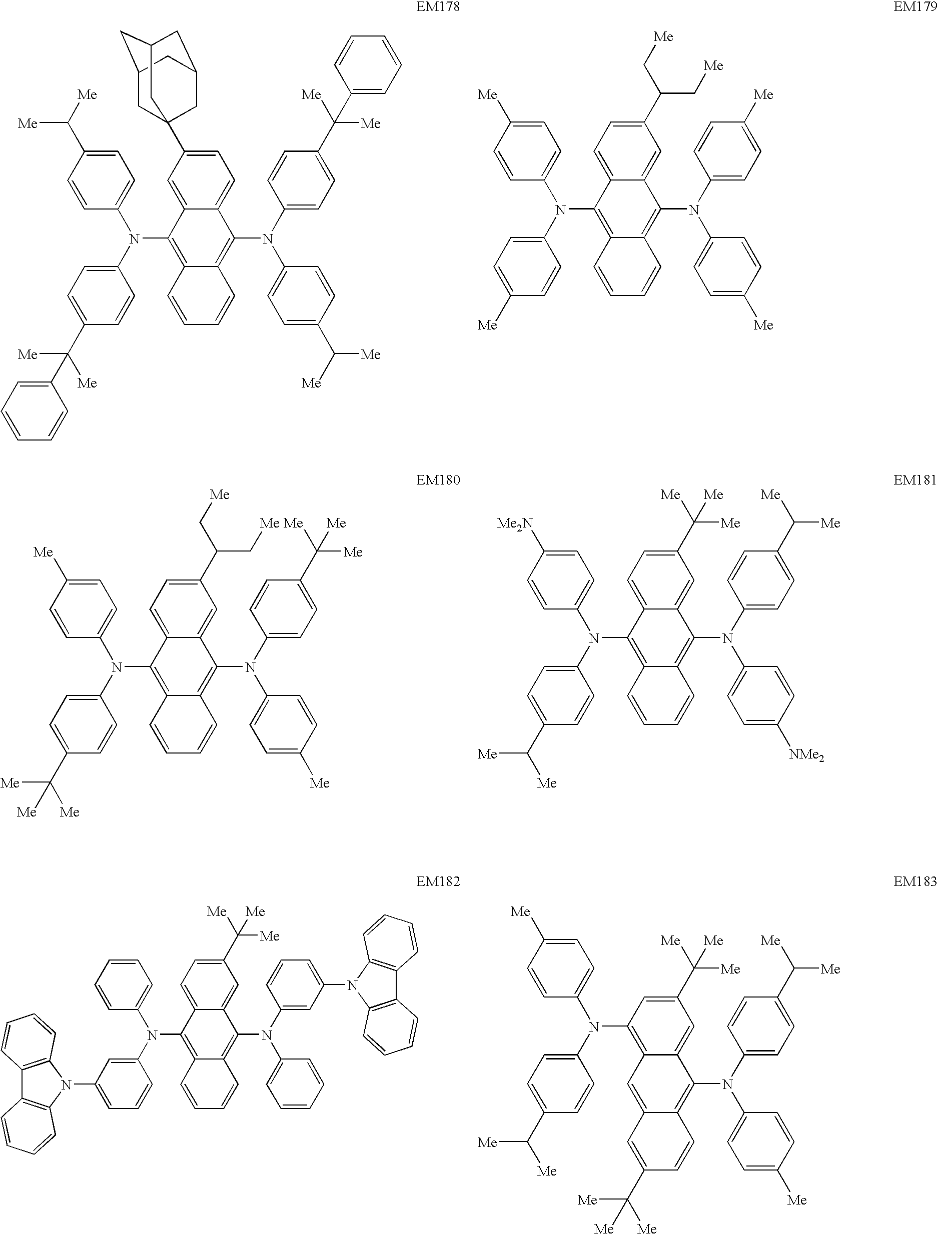

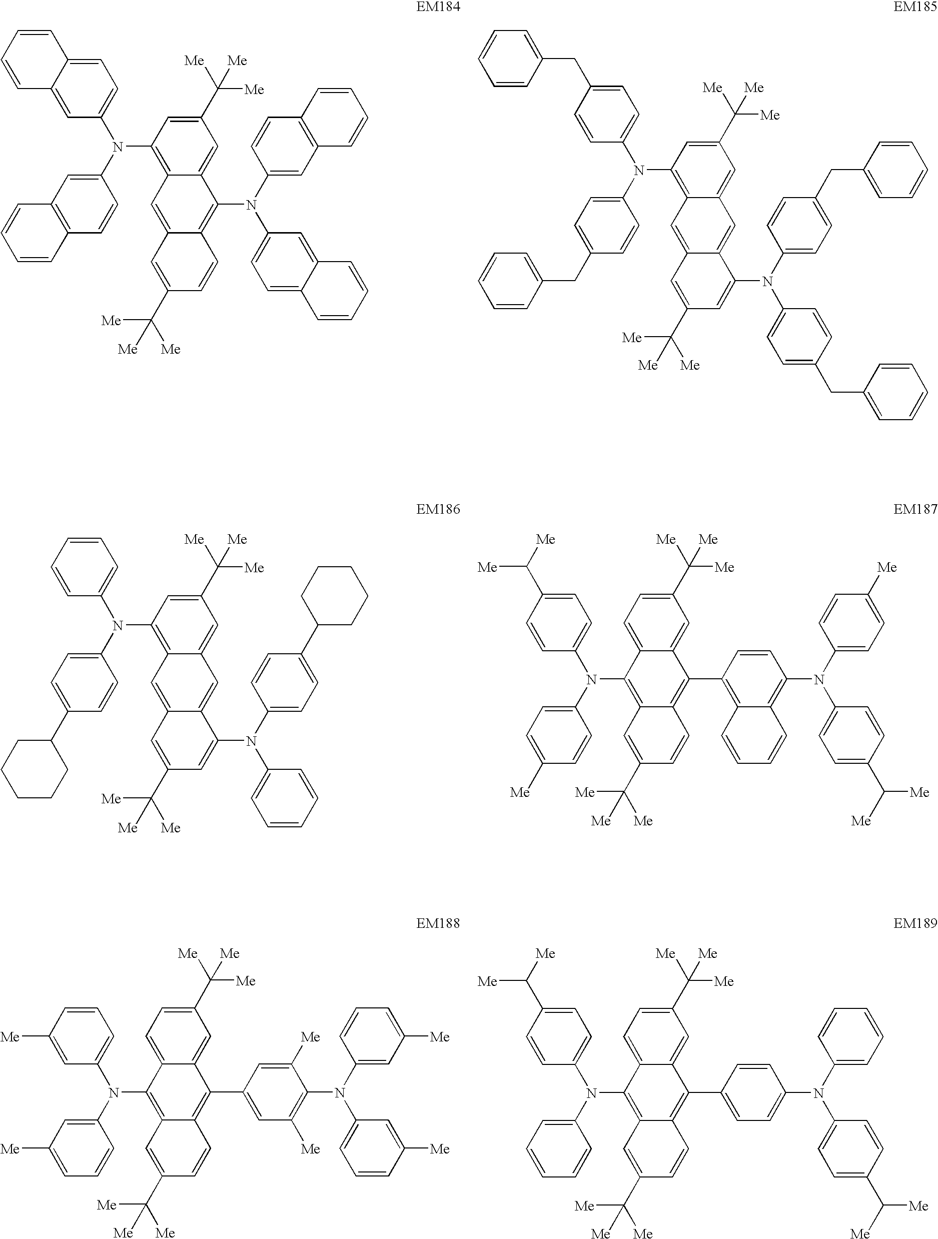

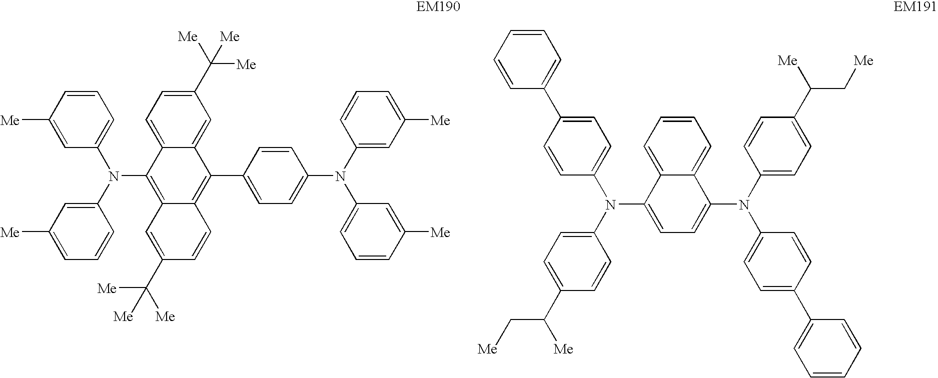

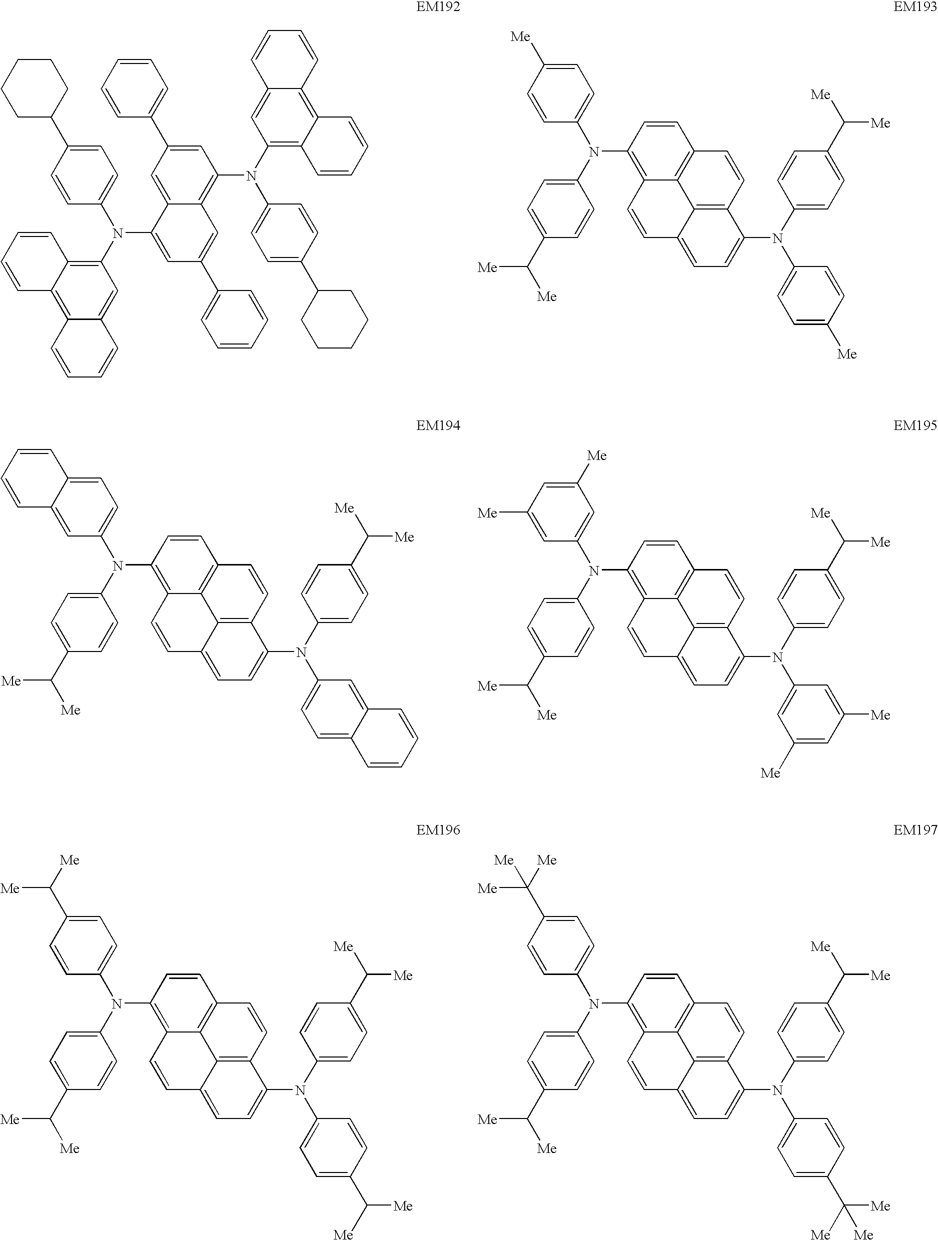

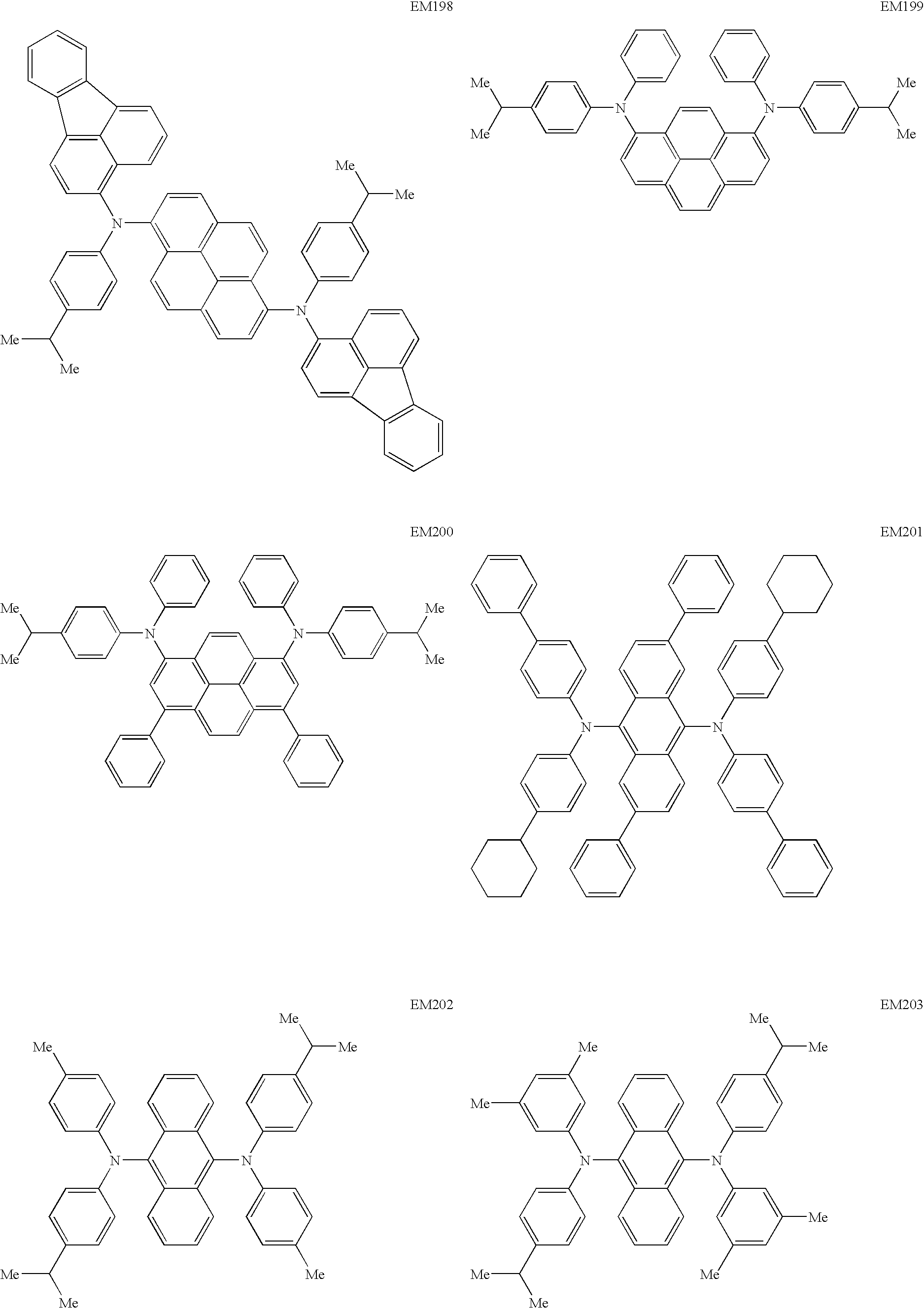

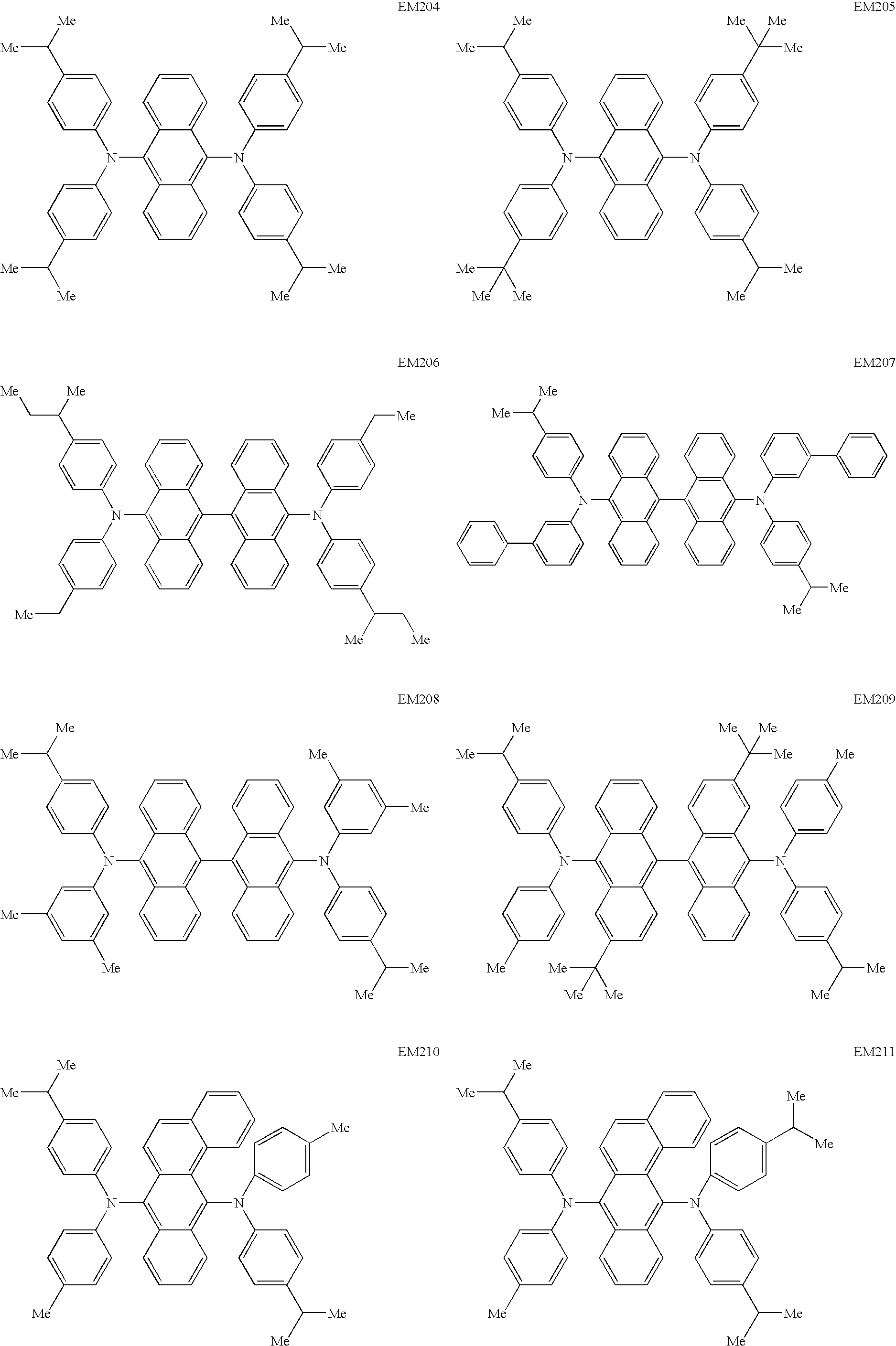

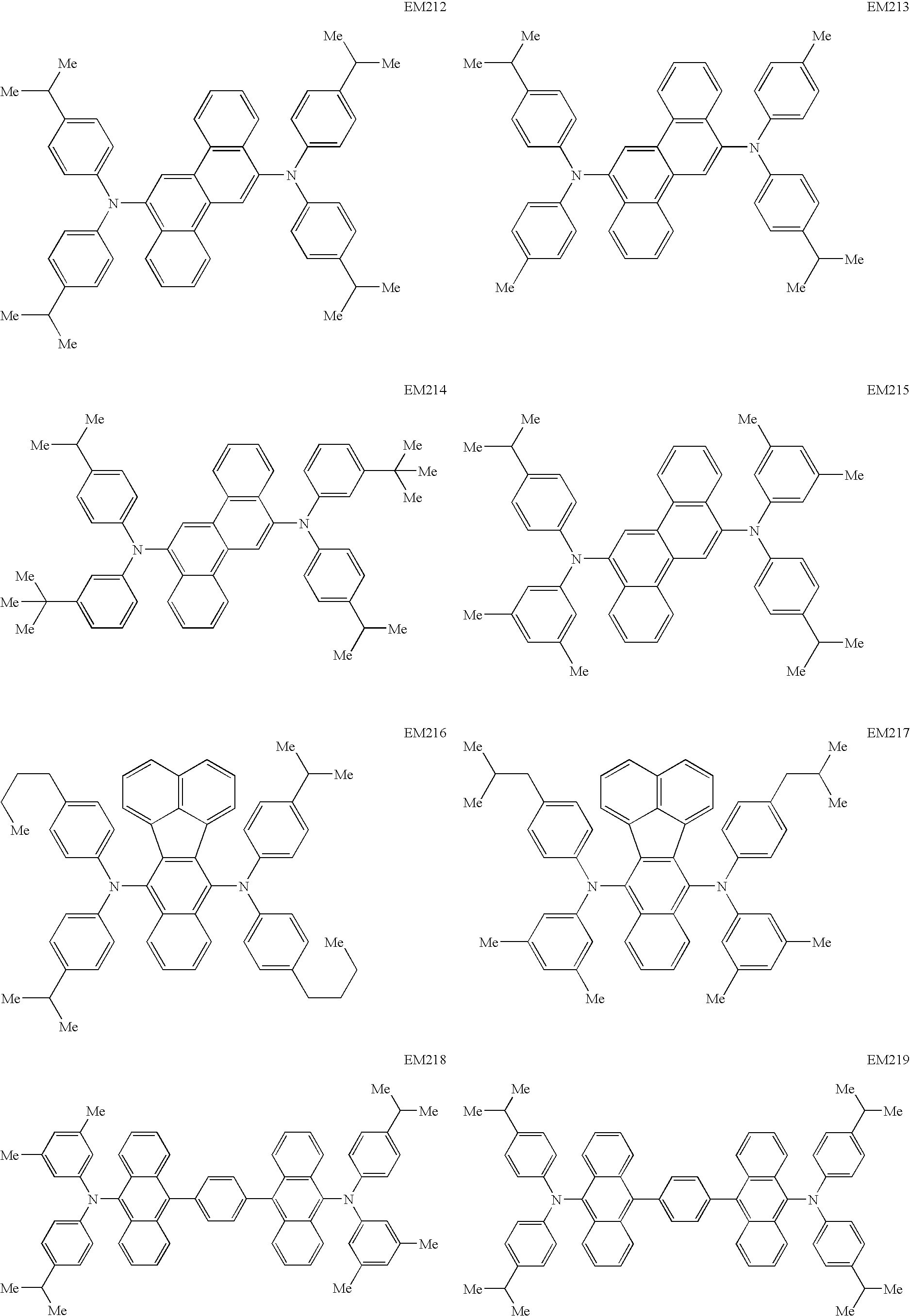

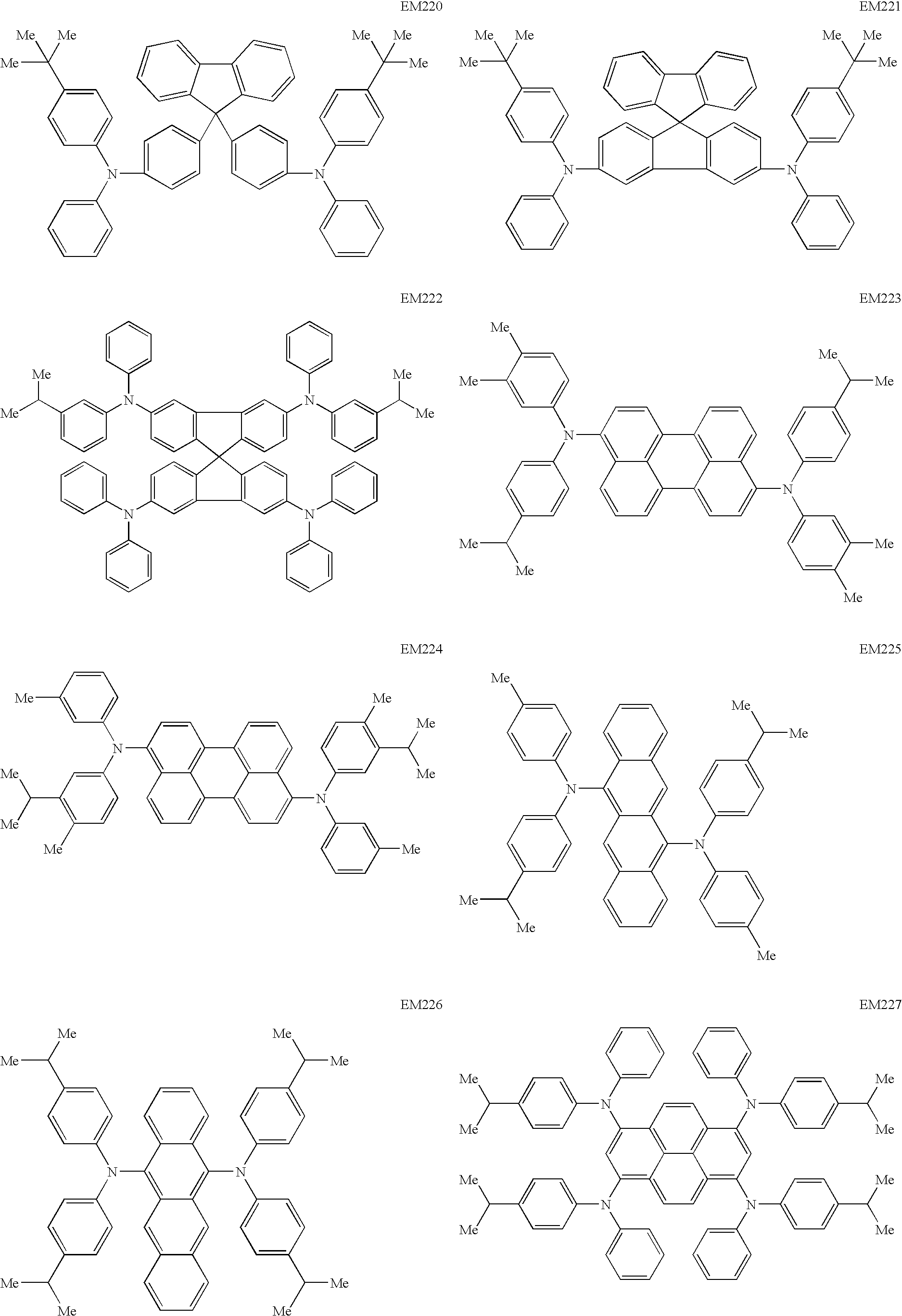

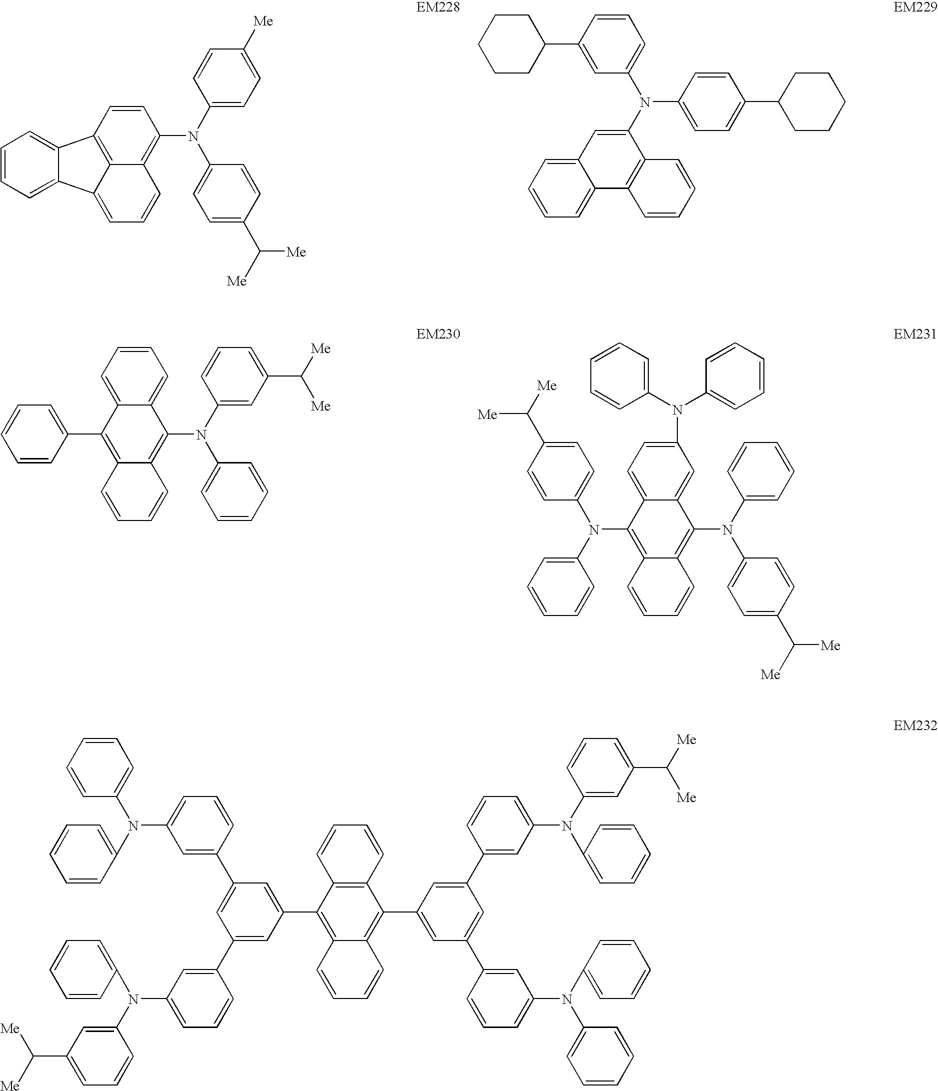

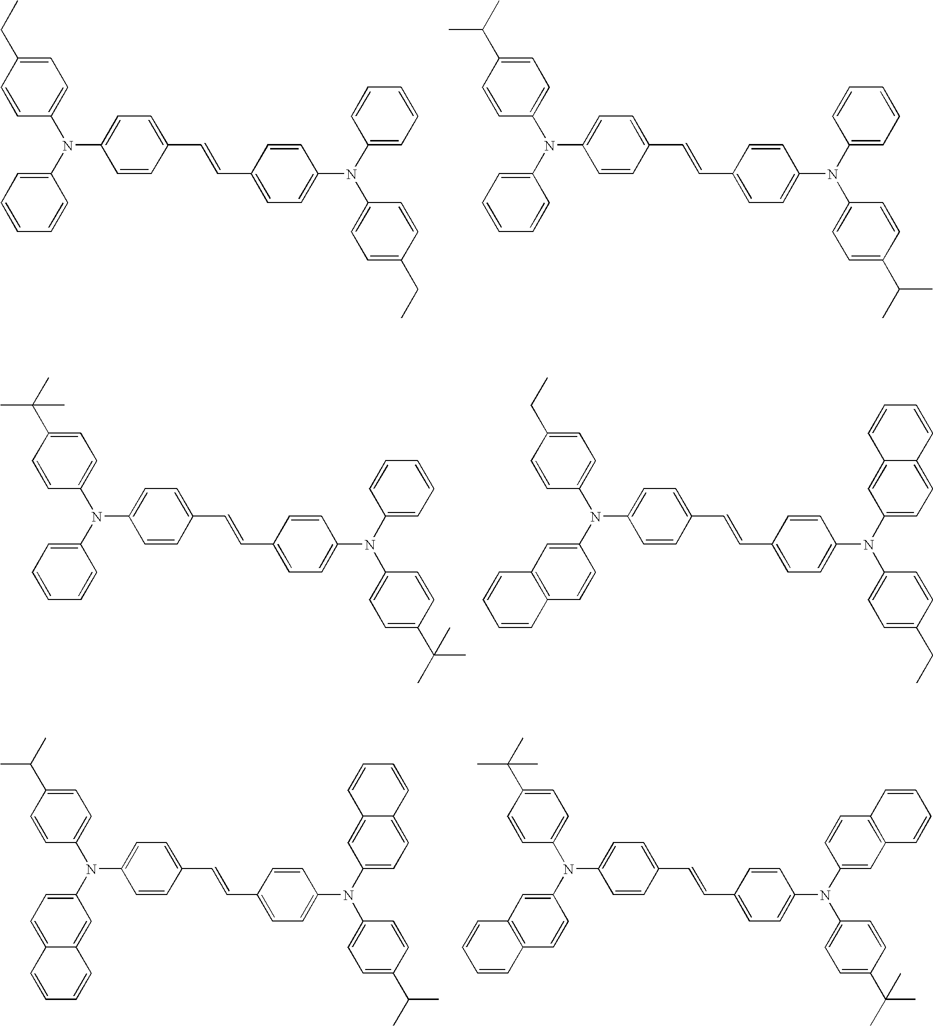

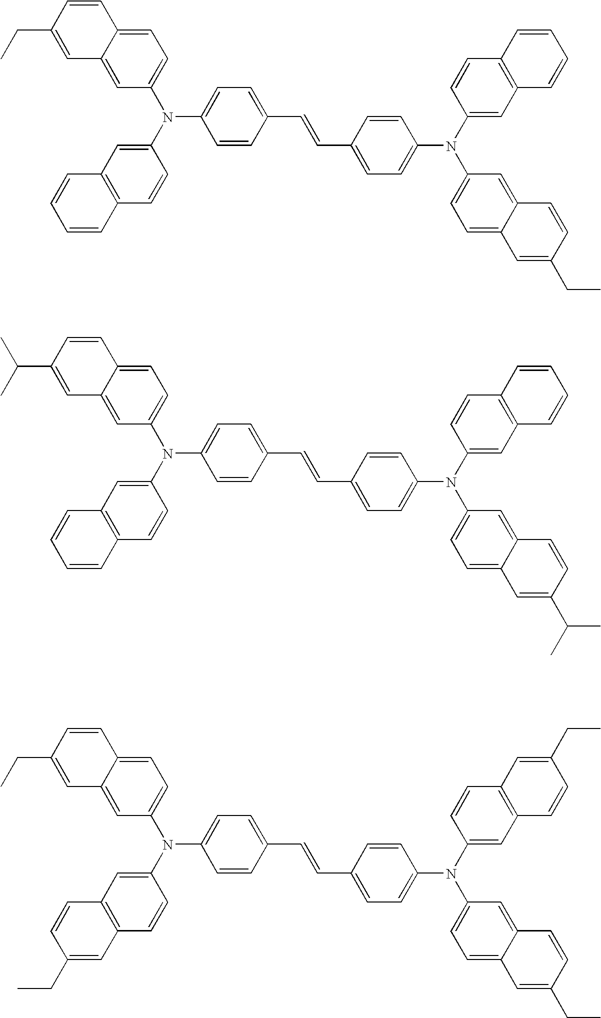

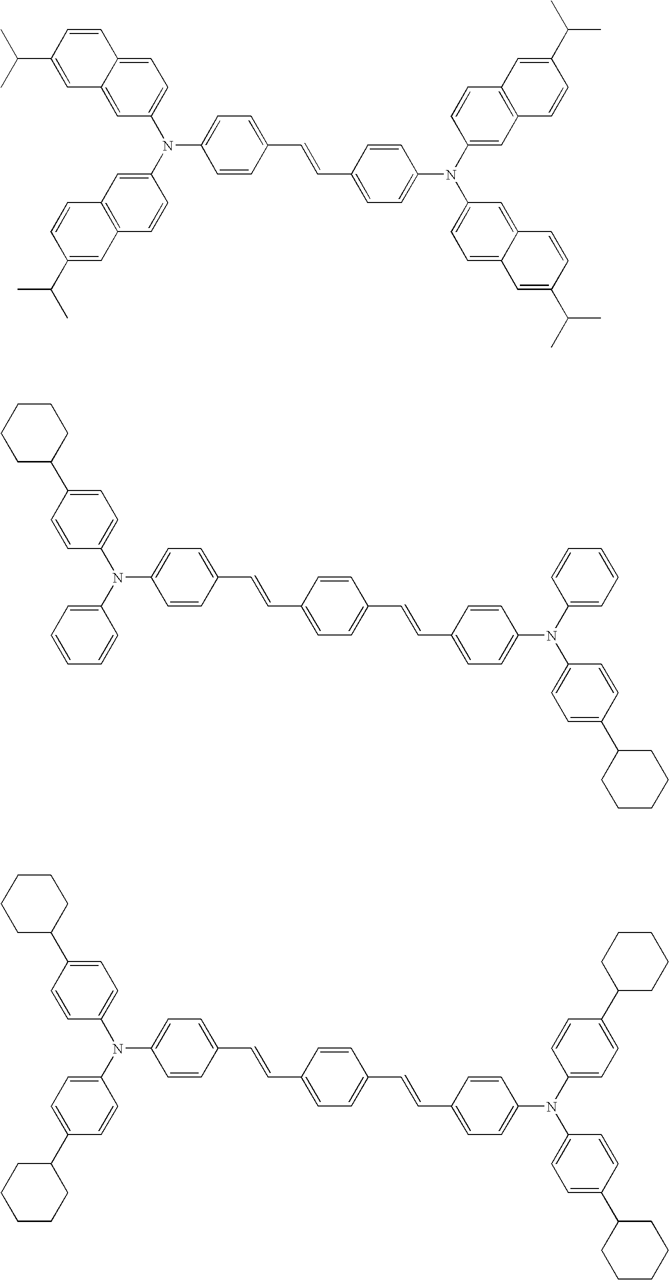

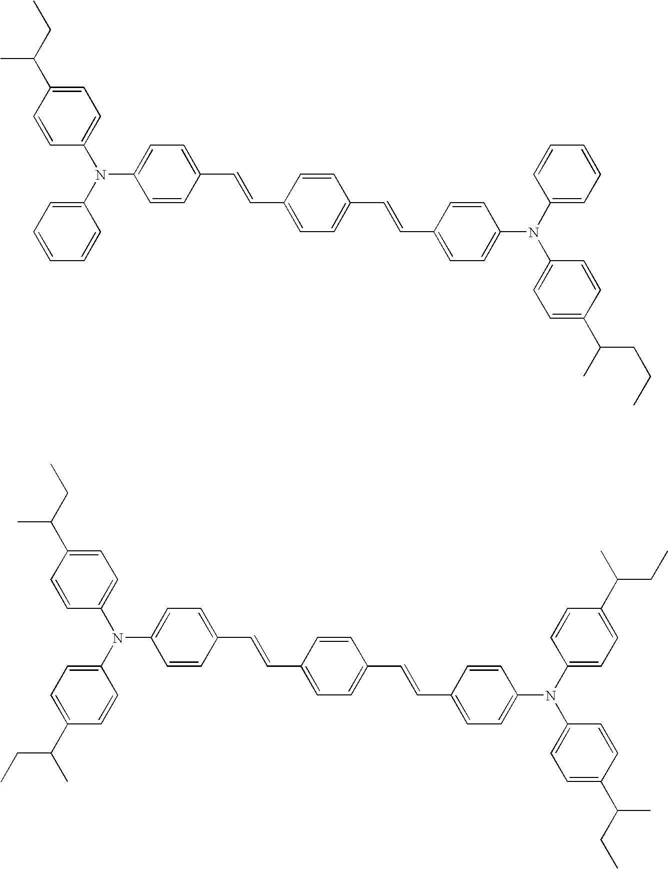

The solvent is a mixed solution of the solvent and the viscosity control agent. The solvent is selected from the group consisting of an aromatic solvent, a halogen type solvent and an ether type solvent. The viscosity control agent is selected from the group consisting of an alcohol type solution, a ketone type solution, a paraffin type solution and an alkyl-substituted aromatic solution having 4 or more carbon atoms.

The solvent is preferably the aromatic solvent, while the viscosity control agent is preferably the alcohol type solution or the alkyl-substituted aromatic solution having 4 or more carbon atoms.

The aromatic solvent as the solvent is more preferably toluene, xylene, mesitylene and chlorobenzene.

The alcohol type solution as the viscosity control agent is a straight-chained or branched alcohol having 1 to 20 carbon atoms, which may be exemplified by methanol, ethanol, propanol, butanol, pentanol, hexanol, heptanol, octanol, nonanol, decanol, a benzyl alcohol derivative and a hydroxyalkyl benzene derivative.

The alkyl-substituted aromatic solution having 4 or more carbon atoms may be exemplified by an alkyl benzene derivative having 4 or more carbon atoms, examples of which include a straight-chained or branched butylbenzene, dodecylbenzene, tetralin and cyclohexylbenzene.

A halogenated hydrocarbon solvent (the halogen type solvent) may be exemplified by dichloromethane, dichloroethane, chloroform, carbon tetrachloride, tetrachloroethane, trichloroethane, chlorobenzene, dichlorobenzene and chlorotoluene.

The ether type solvent may be exemplified by dibutyl ether, tetrahydrofuran, dioxane and anisole.

Now, examples and comparisons of the present invention will be described.

### (Solubility Evaluation)

A description about solubility evaluation will be given below.

(Solubility Evaluation 1)

First, an example of solubility evaluation of compounds used as the host material will be described as Solubility Evaluation 1.

Solubility was evaluated for Compound H1 to Compound H9.

Solubility Evaluation 1 was conducted by the following method.

A hundred mg of each compound was placed in a sample pot and the toluene as the solvent was added until the compound was dissolved. The solubility in the toluene was obtained from an amount of the added toluene.

Solubility Evaluation 1 was conducted on Compounds H1 to H9 shown below.

The result of Solubility Evaluation 1 is shown in Table 1.

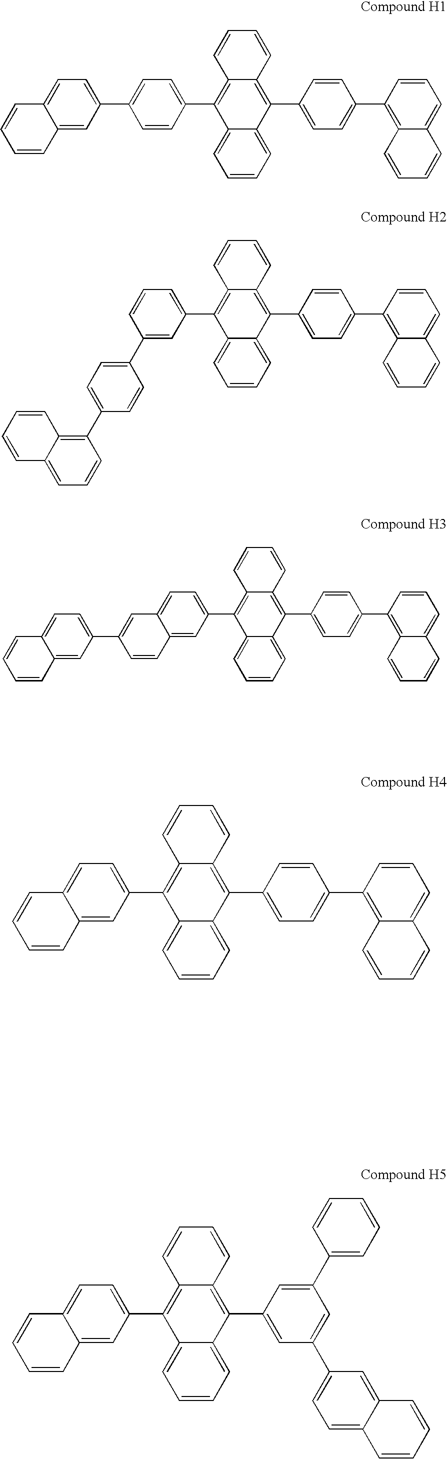

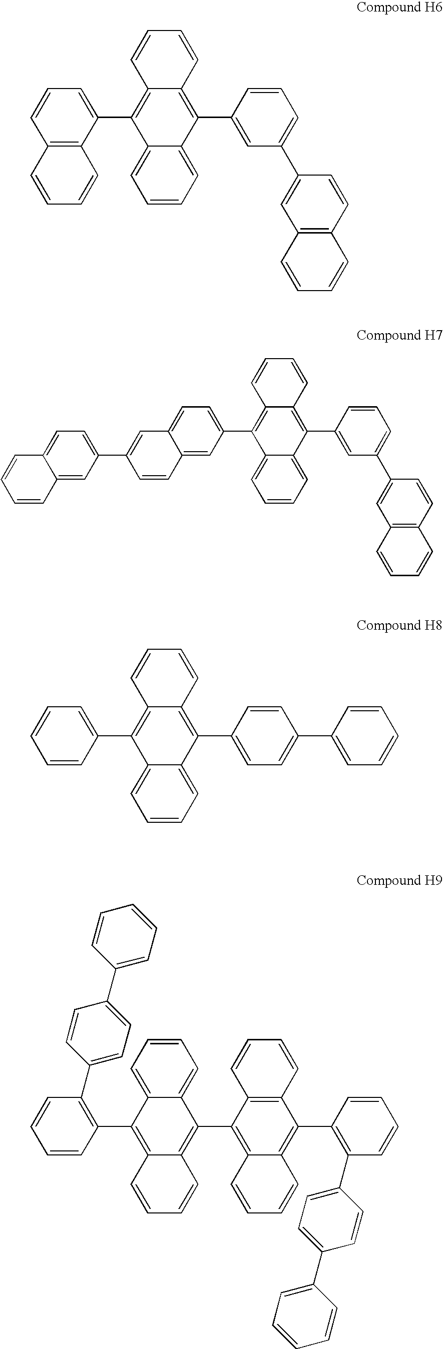

Compounds H1 to H5 showed high solubility in the toluene.

On the other hand, Compounds H6 to H8 showed low solubility of 0.5 wt % or lower.

With the solubility of 0.5 wt % or lower, thickness control of films will be difficult in a wet film formation method, so that Compounds H6, H7 and H8 are not suitable for the wet film formation method.

Compound H9 showed solubility capable of performing the thickness control of the films in the wet film formation method.

The compounds that showed solubility of 0.5 wt % or higher were subjected to an evaluation of a mixed solution ink.

From Solubility Evaluation 1, it was recognized that a specific substituent group is required in order to enhance the solubility of the anthracene compounds.

The results of Compounds H1 to H4 showed that the solubility can be enhanced by bonding a naphthyl group to a central anthracene skeleton in a para position with a phenyl group interposed therebetween, namely, the solubility can be enhanced by Formula (2).

The result of Compound 5 showed that two substituent groups are preferably attached to the phenyl group bonded to the central anthracene skeleton. That is, it was verified that the solubility can be enhanced by Formula (1).

In addition, it was verified that both the two substituent groups attached to the phenyl group are preferably attached in meta positions and that the solubility can be enhanced by Formula (3).

(Solubility Evaluation 2)

Next, an example of solubility evaluation of compounds used as the dopant material will be described as Solubility Evaluation 2.

Solubility Evaluation 2 was conducted on the examples of the compounds described above and Compounds D1 to D4 shown below.

The solubility was obtained by the method same as Solubility Evaluation 1 except that the compounds shown below were used.

The result is shown in Table 2 and Table 3.

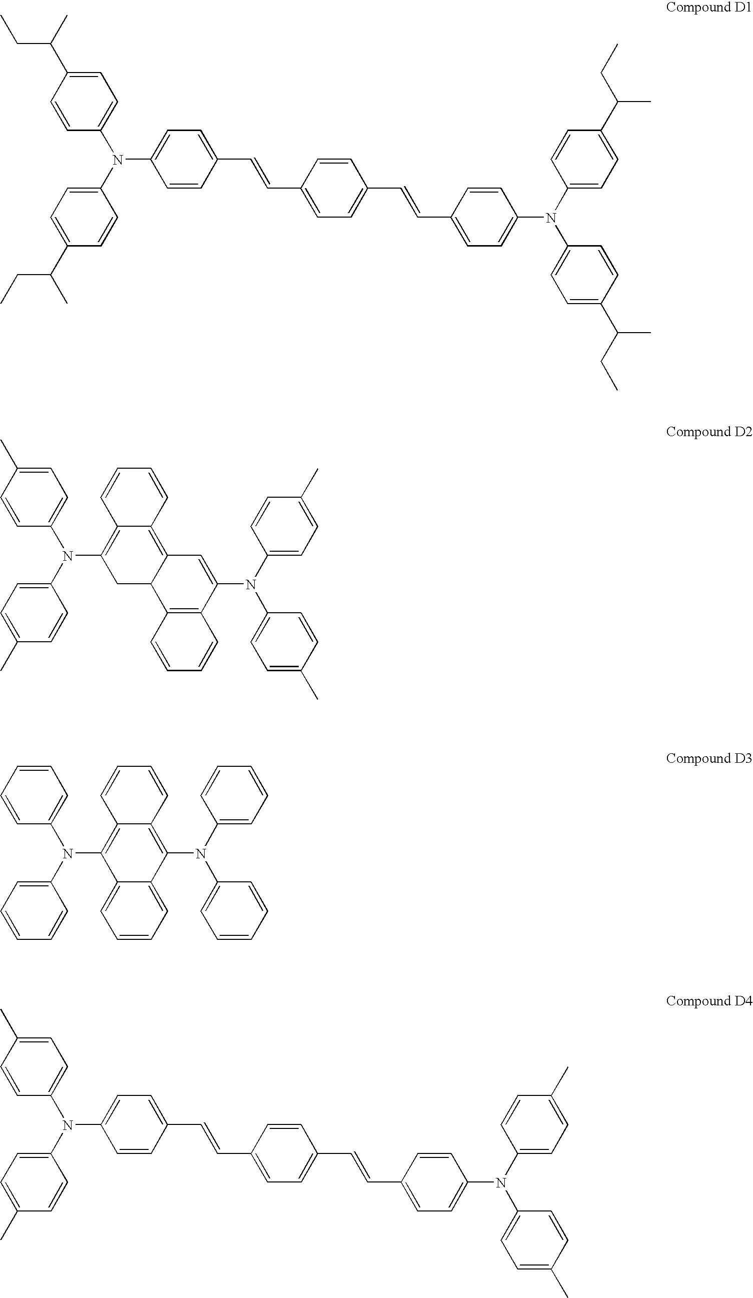

TABLE 1

| | compound | compound | compound | compound | compound | compound | compound | compound | compound |
|---|---|---|---|---|---|---|---|---|---|
| | H1 | H2 | H3 | H4 | H5 | H6 | H7 | H8 | H9 |
| Solubility | 2.0 | 2.0 | 2.0 | 5.0 | 5.0 | 0.25 | 0.25 | 0.2 | 1.4 |
| (%) | | | | | | | | | |

TABLE 2

| | Solubility |
|---|---|
| Compound | (%) |
| EM136 | 0.5 |
| EM137 | 1 |
| EM138 | 0.5 |
| EM139 | 0.5 |
| EM140 | 0.5 |
| EM141 | 0.5 |
| EM142 | 0.5 |
| EM143 | 0.5 |
| EM144 | 0.5 |
| EM145 | 0.5 |
| EM146 | 0.5 |
| EM147 | 1 |
| EM148 | 0.5 |
| EM149 | 0.5 |
| EM150 | 0.5 |
| EM151 | 0.5 |
| EM152 | 1 |
| EM153 | 0.5 |
| EM154 | 0.5 |
| EM155 | 0.5 |
| EM174 | 0.5 |
| EM175 | 0.5 |
| EM176 | 0.5 |
| EM177 | 0.5 |
| EM178 | 0.5 |

TABLE 3

| | Solubility |
|---|---|
| Compound | (%) |
| EM191 | 1 |
| EM192 | 1 |
| EM193 | 0.5 |
| EM194 | 1 |
| EM195 | 0.5 |
| EM196 | 1 |
| EM197 | 1 |
| EM198 | 0.5 |
| EM199 | 0.5 |
| EM200 | 0.5 |
| EM201 | 0.5 |
| EM202 | 0.5 |
| EM203 | 0.5 |
| EM204 | 0.5 |
| EM205 | 0.5 |
| EM206 | 0.5 |
| EM207 | 0.5 |
| EM208 | 1 |
| EM209 | 0.5 |
| EM210 | 1 |
| EM211 | 1 |
| EM212 | 1 |
| EM213 | 0.5 |
| EM214 | 1 |
| EM215 | 1 |

As compared to Compounds D2 to D4, the compounds having specific substituent group were verified to have high solubility in the toluene.

In other words, the branched alkyl group having 2 to 6 carbon atoms or the cycloalkyl group having 5 to 10 carbon atoms are preferable as the substituent group.

### EXAMPLE

Next, inks prepared as examples of the organic EL material-containing solution will be described.

### Examples 1 to 46

Preparation of inks (Ink 1 to Ink 46) was performed as follows.

Specifically, samples of a host compound and a dopant compound were blended in a weight ratio of 20:1 in a sample pot, to which a solvent and a viscosity control agent were added.

The result is shown in Table 5 and Table 6.

Table 5 and Table 6 each show a type of solvent, solid content (wt %), solubility (o: no insoluble matter visually observed, x: insoluble matter visually observed), viscosity and a solution condition after a week.

In Examples 1 to 46, preferable compounds of the present invention were used as the host and the dopant, the toluene (the aromatic solvent) was used as the solvent, and the alcohol type solutions were used as the viscosity control agent.

Examples 1 to 46 showed good solubility, viscosity and pot life.

(Comparison 1)

An ink was prepared using only the toluene as the solvent.

The result is shown in Table 6 as Ink 47.

Although an insoluble matter was not observed, the viscosity was 0.65 cp, which was insufficient for the coating process.

(Comparison 2)

An ink having a solid content of 0.5 wt % was tried to be prepared using Compound H9 as the host and a mixed solution of the toluene and 1-octyl alcohol (mixing ratio=1:1) without using the dopant (Ink 48).

However, the solid could not be dissolved completely and nonuniform dissolving condition was visually observed.

The result showed that even when the host material having a certain level of solubility (0.5 wt %) was used, sufficient solubility could not be secured as the ink to which the viscosity control agent (e.g., alcohol type solution) was added.

(Comparisons 3 to 5)

Inks were each prepared by dissolving Compound H4 as the host and each of Compounds D8 to D10 as the dopant in a mixed solution of the toluene and the 1-octyl alcohol (mixing ratio=1:3). The result is shown in Table 6 as Inks 49 to 51.

No insoluble matter was observed in the inks and the viscosities were in the range from 3 to 3.1 cp.

However, precipitation of the solid was observed within a week.

In other words, it was verified that the dopant also requires to have a predetermined solubility in order to secure sufficient pot life.

TABLE 4

| Compound | Solubility (%) |
|---|---|
| EM175 | 0.5 |
| EM176 | 0.5 |
| EM177 | 0.5 |
| EM178 | 0.5 |
| EM179 | 0.5 |
| EM180 | 1.0 |
| EM181 | 0.5 |
| EM182 | 0.5 |
| EM183 | 0.5 |
| EM184 | 0.5 |
| Compound D1 | 0.5 |
| Compound D2 | <0.1 |
| Compound D3 | <0.1 |
| Compound D4 | <0.1 |

TABLE 5

| | | | | | | | Solution |
|---|---|---|---|---|---|---|---|
| | | | | | | | condition |
| | | | | Solid | | Viscosity | after 1 |
| Ink | Host | Dopant | Solvent (mixing ratio) | content | Solubility | (cP) | week |
| Ink1 | Compound1 | EM-138 | Toluene/1-octyl alcohol | 0.5 | ∘ | 1.6 | No |
| | | | (1:1) | | | | change |
| Ink2 | Compound2 | EM-140 | Toluene/1-octyl alcohol | 0.5 | ∘ | 1.5 | No |
| | | | (1:1) | | | | change |
| Ink3 | Compound3 | EM-148 | Toluene/1-octyl alcohol | 0.5 | ∘ | 1.5 | No |
| | | | (1:1) | | | | change |
| Ink4 | Compound4 | EM-151 | Toluene/1-octyl alcohol | 0.5 | ∘ | 1.5 | No |
| | | | (1:1) | | | | change |
| Ink5 | Compound5 | EM-154 | Toluene/1-octyl alcohol | 0.5 | ∘ | 1.5 | No |
| | | | (1:1) | | | | change |
| Ink6 | Compound1 | EM-174 | Toluene/1-octyl alcohol | 0.5 | ∘ | 1.5 | No |
| | | | (1:1) | | | | change |
| Ink7 | Compound1 | EM-180 | Toluene/1-octyl alcohol | 0.5 | ∘ | 1.5 | No |
| | | | (1:1) | | | | change |
| Ink8 | Compound1 | EM-181 | Toluene/1-octyl alcohol | 0.5 | ∘ | 1.5 | No |
| | | | (1:1) | | | | change |
| Ink9 | Compound1 | EM-184 | Toluene/1-octyl alcohol | 0.5 | ∘ | 1.6 | No |
| | | | (1:1) | | | | change |
| Ink10 | Compound1 | EM-212 | Toluene/1-octyl alcohol | 0.5 | ∘ | 1.5 | No |
| | | | (1:1) | | | | change |
| Ink11 | Compound1 | EM-213 | Toluene/1-octyl alcohol | 0.5 | ∘ | 1.5 | No |
| | | | (1:1) | | | | change |
| Ink12 | Compound1 | EM-214 | Toluene/1-octyl alcohol | 0.5 | ∘ | 1.5 | No |
| | | | (1:1) | | | | change |
| Ink13 | Compound1 | EM-138 | Toluene/2-ethylhexyl | 0.5 | ∘ | 1.5 | No |
| | | | alcohol (1:1) | | | | change |
| Ink14 | Compound1 | EM-141 | Toluene/2-ethylhexyl | 0.5 | ∘ | 1.5 | No |
| | | | alcohol (1:1) | | | | change |
| Ink15 | Compound1 | EM-142 | Toluene/2-ethylhexyl | 0.5 | ∘ | 1.5 | No |
| | | | alcohol (1:1) | | | | change |
| Ink16 | Compound1 | EM-143 | Toluene/2-ethylhexyl | 0.5 | ∘ | 1.5 | No |
| | | | alcohol (1:1) | | | | change |
| Ink17 | Compound4 | EM-144 | Toluene/2-ethylhexyl | 0.5 | ∘ | 1.5 | No |
| | | | alcohol (1:1) | | | | change |
| Ink18 | Compound4 | EM-145 | Toluene/2-ethylhexyl | 0.5 | ∘ | 1.5 | No |
| | | | alcohol (1:1) | | | | change |
| Ink19 | Compound4 | EM-146 | Toluene/2-ethylhexyl | 0.5 | ∘ | 1.5 | No |
| | | | alcohol (1:1) | | | | change |
| Ink20 | Compound4 | EM-147 | Toluene/2-ethylhexyl | 0.5 | ∘ | 1.5 | No |
| | | | alcohol (1:1) | | | | change |
| Ink21 | Compound4 | EM-154 | Toluene/2-ethylhexyl | 0.5 | ∘ | 1.5 | No |
| | | | alcohol (1:1) | | | | change |
| Ink22 | Compound4 | EM-180 | Toluene/2-ethylhexyl | 0.5 | ∘ | 1.5 | No |
| | | | alcohol (1:1) | | | | change |
| Ink23 | Compound4 | EM-213 | Toluene/2-ethylhexyl | 0.5 | ∘ | 1.5 | No |
| | | | alcohol (1:1) | | | | change |
| Ink24 | Compound4 | EM-214 | Toluene/2-ethylhexyl | 0.5 | ∘ | 1.5 | No |
| | | | alcohol (1:1) | | | | change |
| Ink25 | Compound4 | EM-215 | Toluene/2-ethylhexyl | 0.5 | ∘ | 1.5 | No |
| | | | alcohol (1:1) | | | | change |

TABLE 6

| | | | | | | | Solution |
|---|---|---|---|---|---|---|---|
| | | | | Solid | | | condition |
| | | | | content | | Viscosity | after 1 |
| Ink | Host | Dopant | Solvent | (wt %) | Solubility | (cP) | week |
| Ink26 | Compound4 | EM-191 | Toluene/1-octyl | 1 | ∘ | 1.6 | No |
| | | | alcohol (1:1) | | | | change |
| Ink27 | Compound4 | EM-192 | Toluene/1-octyl | 1 | ∘ | 1.5 | No |
| | | | alcohol (1:1) | | | | change |
| Ink28 | Compound4 | EM-193 | Toluene/1-octyl | 1 | ∘ | 1.5 | No |
| | | | alcohol (1:1) | | | | change |
| Ink29 | Compound4 | EM-194 | Toluene/1-octyl | 1 | ∘ | 1.5 | No |
| | | | alcohol (1:1) | | | | change |
| Ink30 | Compound4 | EM-195 | Toluene/1-octyl | 1 | ∘ | 1.5 | No |
| | | | alcohol (1:1) | | | | change |
| Ink31 | Compound4 | EM-196 | Toluene/1-octyl | 1 | ∘ | 1.6 | No |
| | | | alcohol (1:1) | | | | change |
| Ink32 | Compound4 | EM-197 | Toluene/1-octyl | 1 | ∘ | 1.5 | No |
| | | | alcohol (1:1) | | | | change |
| Ink33 | Compound4 | EM-198 | Toluene/1-octyl | 1 | ∘ | 1.5 | No |
| | | | alcohol (1:1) | | | | change |
| Ink34 | Compound4 | EM-199 | Toluene/1-octyl | 1 | ∘ | 1.6 | No |
| | | | alcohol (1:1) | | | | change |
| Ink35 | Compound4 | EM-200 | Toluene/1-octyl | 1 | ∘ | 1.5 | No |
| | | | alcohol (1:1) | | | | change |
| Ink36 | Compound4 | EM-213 | Toluene/1-octyl | 0.5 | ∘ | 3.0 | No |
| | | | alcohol (1:3) | | | | change |
| Ink37 | Compound4 | EM-139 | Toluene/1-octyl | 0.5 | ∘ | 3.0 | No |
| | | | alcohol (1:3) | | | | change |
| Ink38 | Compound4 | EM-151 | Toluene/2-ethylhexyl | 0.5 | ∘ | 2.6 | No |
| | | | alcohol (1:3) | | | | change |
| Ink39 | Compound4 | EM-170 | Toluene/2-ethylhexyl | 0.5 | ∘ | 2.7 | No |
| | | | alcohol (1:3) | | | | change |
| Ink40 | Compound4 | EM-171 | Toluene/2-ethylhexyl | 0.5 | ∘ | 2.6 | No |
| | | | alcohol (1:3) | | | | change |
| Ink41 | Compound4 | EM-172 | Toluene/2-ethylhexyl | 0.5 | ∘ | 2.6 | No |
| | | | alcohol (1:3) | | | | change |
| Ink42 | Compound4 | EM-173 | Toluene/2-ethylhexyl | 0.5 | ∘ | 2.6 | No |
| | | | alcohol (1:3) | | | | change |
| Ink43 | Compound4 | EM-190 | Toluene/2-ethylhexyl | 0.5 | ∘ | 2.7 | No |
| | | | alcohol (1:3) | | | | change |
| Ink44 | Compound4 | EM-191 | Toluene/2-ethylhexyl | 0.5 | ∘ | 2.6 | No |
| | | | alcohol (1:3) | | | | change |
| Ink45 | Compound4 | EM-201 | Toluene/2-ethylhexyl | 0.5 | ∘ | 2.6 | No |
| | | | alcohol (1:3) | | | | change |
| Ink46 | Compound4 | EM-203 | Toluene/2-ethylhexyl | 0.5 | ∘ | 2.6 | No |
| | | | alcohol (1:3) | | | | change |
| Ink47 | Compound4 | EM-138 | Toluene | 0.5 | ∘ | 0.65 | No |
| | | | | | | | change |
| Ink48 | Compound9 | — | Toluene/1-octyl | 0.5 | x | — | — |
| | | | alcohol (1:1) | | | | |
| Ink49 | Compound4 | Compound8 | Toluene/1-octyl | 0.5 | ∘ | 3.0 | Precipitate |
| | | | alcohol (1:3) | | | | observed |
| Ink50 | Compound4 | Compound9 | Toluene/1-octyl | 0.5 | ∘ | 3.0 | Precipitate |
| | | | alcohol (1:3) | | | | observed |
| Ink51 | Compound4 | Compound10 | Toluene/1-octyl | 0.5 | ∘ | 3.1 | Precipitate |
| | | | alcohol (1:3) | | | | observed |

From the results above, the following was verified.

### (1) In order to prepare an ink having a high solution viscosity for the process suitability, the viscosity control agent such as the alcohol type solution has to be added to the solvent such as the toluene.

### (2) The host requires a high solubility so as to be dissolvable even in the mixed solution (solvent+viscosity control agent), so that the host needs to have a specific structure.

### (3) In order to prepare an ink with a long pot life, the amine compound used as the dopant needs to have a specific substituent group.

Specifically, it was verified that an organic EL ink having excellent process suitability could be prepared from a combination of the anthracene compound having the specific structure, the amine compound having the specific substituent group and the specific mixed solution.

(Organic EL Device)

Next, an organic EL device will be described.

### (Arrangement of Organic EL Device)

A description about an arrangement of the organic EL device will be given.

### [1] Arrangement of Organic EL Device

Typical arrangement of the organic EL device may be exemplified by the following arrangements.

(1) anode/luminescent layer/cathode
(2) anode/hole injection layer/luminescent layer/cathode
(3) anode/luminescent layer/electron injection layer/cathode
(4) anode/hole injection layer/luminescent layer/electron injection layer/cathode
(5) anode/organic semiconductor layer/luminescent layer/cathode
(6) anode/organic semiconductor layer/electron blocking layer/luminescent layer/cathode
(7) anode/organic semiconductor layer/luminescent layer/adhesion improving layer/cathode
(8) anode/hole injection layer/hole transport layer/luminescent layer/electron injection layer/cathode
(9) anode/insulating layer/luminescent layer/insulating layer/cathode
(10) anode/inorganic semiconductor layer/insulating layer/luminescent layer/insulating layer/cathode
(11) anode/organic semiconductor layer/insulating layer/luminescent layer/insulating layer/cathode
(12) anode/insulating layer/hole injection layer/hole transport layer/luminescent layer/insulating layer/cathode
(13) anode/insulating layer/hole injection layer/hole transport layer/luminescent layer/electron injection layer/cathode

Among these, the arrangement (8) is usually preferable.

### [2] Light-Transmissive Substrate

The organic EL device is formed on a light-transmissive substrate. The light-transmissive substrate used herein is a substrate supporting the organic EL device, which is preferably a flat substrate having a transmittance of 50% or higher for a light in the visible range of 400 to 700 nm.

Specifically, a glass plate, a polymer plate and the like may be employed.

Particularly, the glass plate may include a soda-lime glass, a barium/strontium-containing glass, a lead glass, an aluminosilicate glass, a borosilicate glass, a barium borosilicate glass and quartz.

The polymer plate may include a polycarbonate, an acryl, a polyethylene terephthalate, a polyether sulfide and a polysulfone.

### [3] Anode

The anode of the organic EL device injects a hole in the hole transport layer and the luminescent layer, so that it is efficient that the anode has a work function of 4.5 eV or higher. Concrete examples of an anode material may include indium-tin oxide (ITO), tin oxide (NESA), indium zinc oxide (IZO), gold, silver, platinum and copper. The anode with smaller work function is more preferable in order to inject an electron to the electron transport layer and the luminescent layer.

The anode may be made by forming a thin film from these electrode materials through methods such as vapor deposition and sputtering.

When luminescence from the luminescent layer is taken out from the anode, the anode preferably has a transmittance of higher than 10% for the luminescence. The sheet resistance of the anode is preferably several hundreds Ω/square or lower. The thickness of the anode is typically in the range from 10 nm to 1 μm, and preferably in the range from 10 to 200 nm, though it depends on the material of the anode.

### [4] Luminescent Layer

The luminescent layer of the organic EL device has functions below:

(1) Injecting function: a function for allowing the hole to be injected thereto by the anode or the hole injection layer, or for allowing the electron to be injected thereto by the cathode or the electron injection layer when an electrical field is applied;
(2) Transport function: a function for transporting injected electric charges (the electron and the hole) by the force of the electrical field; and

### (3) Luminescent function; a function for providing a condition for recombination of the electron and the hole to generate luminescence.

Herein, although there may be a difference in degrees of easiness of receiving the injected hole and that of the injected electron and a difference in transporting capabilities represented by mobilities of the hole and the electron, the luminescent layer preferably transports one of the electric charges.

Conventional methods such as vapor deposition, spin coating and an LB method may be employed as a method for forming the luminescent layer.

The luminescent layer is particularly preferably a molecular deposition film.

Here, the molecular deposition film is a thin film that is formed by depositing a material compound in the gas phase or a film formed by solidifying a material compound in a solution state or the liquid phase. The molecular deposition film can be typically distinguished from a thin film formed by the LB method (molecular built-up film) by differences in aggregation structures and higher order structures and differences in resulting functions.

In addition, as disclosed in JP-A-57-51781, the luminescent layer can be formed by preparing a solution by dissolving a binder such as a resin and the material compound in a solvent and forming a thin film from the solution by spin coating or the like.

The thickness of the luminescent layer is preferably in the range from 5 to 50 nm, more preferably in the range from 7 to 50 nm and most preferably in the range 10 to 50 nm. The thickness below 5 nm may cause difficulty in forming the luminescent layer and in controlling chromaticity, while the thickness above 50 nm may raise driving voltage.

### [5] Hole Injection/Transport Layers (Hole Transport Zone)

The hole injection/transport layer helps injection of the hole to the luminescent layer and transport the hole to a luminescent region, in which the hole mobility is large and the energy of ionization is typically small (5.5 eV or smaller). A material of the hole injection/transport layer is preferably those transporting the hole to the luminescent layer with a low field intensity, and more preferably those transporting the hole with the hole mobility of, for example, 10⁴ to 10⁶ V/cm or at least 10⁻⁴ cm²/V·sec when the electrical field is applied.

Concrete examples of the material may include a triazole derivative (see, for instance, the specification of U.S. Pat. No. 3,112,197), an oxadiazole derivative (see, for instance, the specification of U.S. Pat. No. 3,189,447), an imidazole derivative (see, for instance, the publication of JP-B-37-16096), a polyarylalkane derivative (see, for instance, the specifications of U.S. Pat. No. 3,615,402, No. 3,820,989 and No. 3,542,544 and the publications of JP-B-45-555, JP-B-51-10983, JP-A-51-93224, JP-A-55-17105, JP-A-56-4148, JP-A-55-108667, JP-A-55-156953, and JP-A-56-36656), a pyrazoline derivative and a pyrazolone derivative (see, for instance, the specifications of U.S. Pat. No. 3,180,729 and No. 4,278,746 and the publications of JP-A-55-88064, JP-A-55-88065, JP-49-105537, JP-A-55-51086, JP-A-56-80051, JP-A-56-88141, JP-A-57-45545, JP-A-54-112637 and JP-A-55-74546, a phenylenediamine derivative (see, for instance, the specification of U.S. Pat. No. 3,615,404 and the publications of JP-B-51-10105, JP-B-46-3712, JP-B-47-25336, JP-A-54-53435, JP-A-54-110536 and JP-A-54-119925), an arylamine derivative (see, for instance, the specifications of U.S. Pat. No. 3,567,450, No. 3,180,703, No. 3,240,597, No. 3,658,520, No. 4,232,103, No. 4,175,961 and No. 4,012,376 and the publications of JP-B-49-35702, JP-B-39-27577, JP-A-55-144250, JP-A-56-119132 and JP-A-56-22437 and the specification of West Germany Patent No. 1,110,518), an amino-substituted chalcone derivative (see, for instance, the specification of U.S. Pat. No. 3,526,501), an oxazole derivative (disclosed in, for instance, the specification of U.S. Pat. No. 3,257,203), a styrylanthracene derivative (see, for instance, the publication of JP-A-56-46234), a fluorenone derivative (see, for instance, the publication of JP-A-54-110837), a hydrazone derivative (see, for instance, the specification of U.S. Pat. No. 3,717,462 and the publications of JP-A-54-59143, JP-A-55-52063, JP-A-55-52064, JP-A-55-46760, JP-A-55-85495, JP-A-57-11350, JP-A-57-148749 and JP-A-2-311591), a stilbene derivative (see, for instance, the publications of JP-A-61-210363, JP-A-61-228451, JP-A-61-14642, JP-A-61-72255, JP-A-62-47646, JP-A-62-36674, JP-A-62-10652, JP-A-62-30255, JP-A-60-93455, JP-A-60-94462, JP-A-60-174749 and JP-A-60-175052), a silazane derivative (see the specification of U.S. Pat. No. 4,950,950), a polysilane type (see the publication of JP-A-2-204996), an aniline-type copolymer (see the publication of JP-A-2-282263), and a conductive high-molecular oligomer (thiophene oligomer) disclosed in the publication of JP-A-1-211399.

Although the substances listed above can be used as the material of the hole injection/transport layer, it is preferable to use a porphyrin compound (disclosed in, for instance, the publication of JP-A-63-2956965), an aromatic tertiary amine compound and a styrylamine compound (see, for instance, the specification of U.S. Pat. No. 4,127,412 and the publications of JP-A-53-27033, JP-A-54-58445, JP-A-54-149634, JP-A-54-64299, JP-A-55-79450, JP-A-55-144250, JP-A-56-119132, JP-A-61-29558, JP-A-61-98353 and JP-A-63-295695), and among these, the aromatic tertiary amine compound is particularly preferable.

In addition, 4,4′-bis(N-(1-naphthyl)-N-phenylamino)biphenyl (hereinafter, abbreviated as NPD) having in the molecule two condensed aromatic rings disclosed in U.S. Pat. No. 5,061,569, 4,4′,4″-tris(N-3-methylphenyl-N-phenyl-amino)triphenylamine (hereinafter, abbreviated as MTDATA) in which three triphenylamine units disclosed in the publication of JP-A-4-308688 are linked in a starbust form and the like may also be used.

In addition to the aromatic dimethylidine compound mentioned above as the material of the luminescent layer, inorganic compounds such as p-type Si and p-type SiC can be used as the material of the hole injection layer.

The hole injection/transport layer can be made by forming thin films from the compounds listed above by conventional methods such as the vacuum deposition, the spin coating, a casting method and the LB method.

The thickness of the hole injection/transport layer is not particularly limited, but typically in the range from 5 nm to 5 μm.

### [6] Electron Injection/Transport Layer (Electron Transport Zone)

The electron injection/transport layer may further be laminated between the organic luminescent layer and the cathode. The electron injection/transport layer helps injection of the electron to the luminescent layer and have a high electron mobility.

It is known that, in the organic EL, since light emitted by the organic EL is reflected by an electrode (the cathode, in this case), light directly taken out from the anode and the light taken out after being reflected by the electrode interfere with each other. In order to efficiently utilize the interference, the thickness of the electron transport layer is properly selected from the range of several nanometers to several micrometers. However, especially when the thickness of the layer is large, it is preferable that the electron mobility is at least 10⁻⁵ cm²/Vs or higher under the condition where the electrical field of 10⁴ to 10⁵ Vcm is applied to prevent voltage rise.

As a material used for the electron injection/transport layer, 8-hydroxyquinoline or a metal complex of its derivative is preferable.

Concrete examples of the 8-hydroxyquinoline or the metal complex of its derivative may include metal chelate oxynoid compounds including a chelate of oxine (typically 8-quinolinol or 8-hydroxyquinoline). For example, Alq having A1 as its central metal can be used for the electron injection/transport layer.

An oxadiazole derivative represented by the formula below is also preferable as a material of the electron injection (transport) layer.

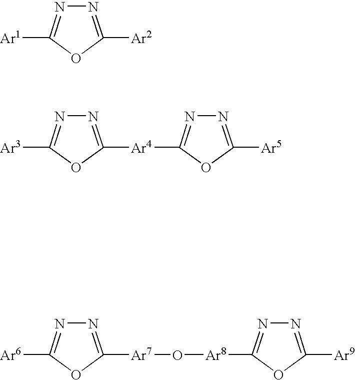

(In the formula, Ar¹,Ar²,Ar³,Ar⁵,Ar⁶ and Ar⁹ each represent a substituted or unsubstituted aryl group, which may be the same or different from each other. Ar⁴,Ar⁷ and Ar⁸ each represent a substituted or unsubstituted arylene group, which may be the same or different from each other.

The aryl group may include a phenyl group, a biphenyl group, an anthranil group, a perylenyl group, and a pyrenyl group. The arylene group may include a phenylene group, a naphtylene group, a biphenylene group, an anthranylene group, a perylenylene group and a pyrenylene group. The substituent group may include an alkyl group having 1 to 10 carbon atoms, an alkoxy group having 1 to 10 carbon atoms and a cyano group. The electron transport compounds are preferably those exhibiting good performance in forming a thin film.

Concrete examples of the electron transport compounds may include substances below.

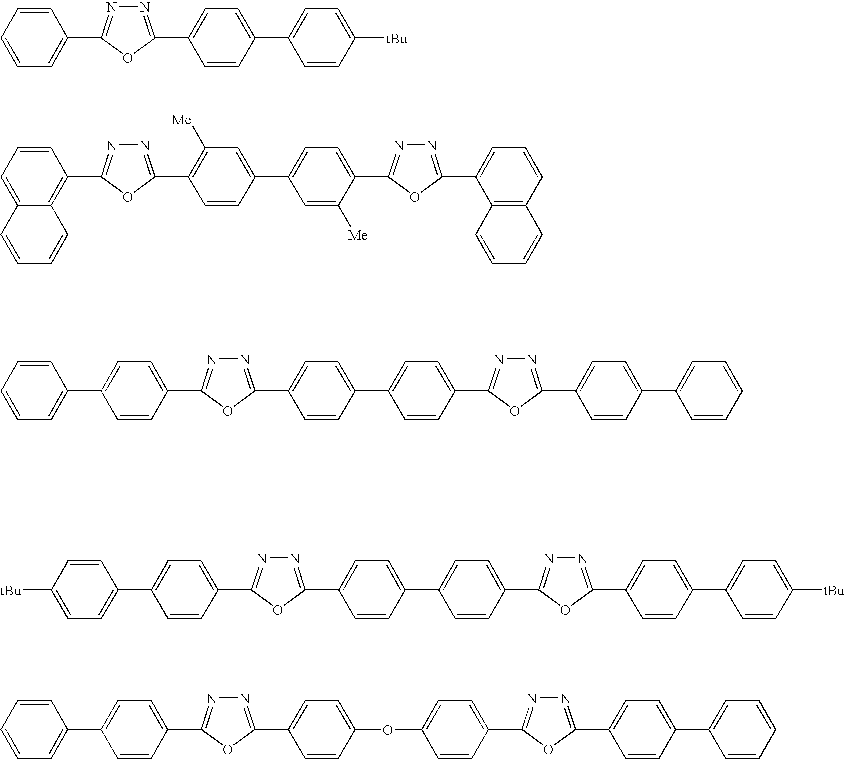

A nitrogen-containing heterocycle derivative represented by the formula below is also preferable as a material of the electron injection (transport) layer.

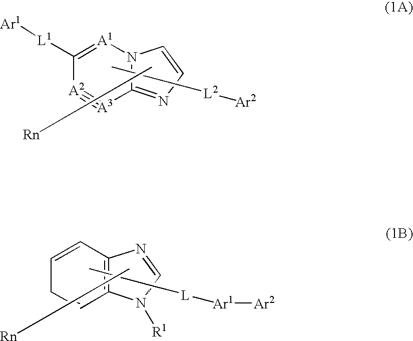

In the formula, A¹ to A³ each represent a nitrogen atom or a carbon atom; R represents an aryl group having 6 to 60 carbon atoms which may have a substituent group, a heteroaryl group having 3 to 60 carbon atoms which may have a substituent group, an alkyl group having 1 to 20 carbon atoms, a haloalkyl group having 1 to 20 carbon atoms or an alkoxy group having 1 to 20 carbon atoms; and n represents an integer of 0 to 5, where the plurality of R may be the same or different from each other when n is an integer equal to or larger than two. In addition, a plurality of adjacent R may be bonded to each other to form a substituted or unsubstituted carbocyclic aliphatic ring or a substituted or unsubstituted carbocyclic aromatic ring. Ar¹ represents the aryl group having 6 to 60 carbon atoms which may have the substituent group or the heteroaryl group having 3 to 60 carbon atoms which may have the substituent group; and Ar² represents a hydrogen atom, the alkyl group having 1 to 20 carbon atoms, the haloalkyl group having 1 to 20 carbon atoms, the alkoxy group having 1 to 20 carbon atoms, the aryl group having 6 to 60 carbon atoms which may have the substituent group or the heteroaryl group having 3 to 60 carbon atoms which may have the substituent group, one of Ar¹ and Ar² being a condensed ring group having 10 to 60 carbon atoms which may have a substituent group or a condensed heterocyclic group having 3 to 60 carbon atoms which may have a substituent group. L¹ and L² each represent a single bond, a condensed ring having 6 to 60 carbon atoms which may have a substituent group, a condensed heterocycle having 3 to 60 carbon atoms which may have a substituent group or a fluorenylene group which may have a substituent group.)

HAr-L¹-Ar¹-Ar²

(In the formula, HAr represents a nitrogen-containing ring having 3 to 40 carbon atoms which may have a substituent group; L¹ represents a single-bonded arylene group having 6 to 60 carbon atoms which may have a substituent group, a heteroarylene group having 3 to 60 carbon atoms which may have a substituent group or a fluorenylene group which may have a substituent group; Ar¹ represents a divalent aromatic hydrocarbon group having 6 to 60 carbon atoms which may have a substituent group; and Ar² represents an aryl group having 6 to 60 carbon atoms which may have a substituent group, a heteroaryl group having 3 to 60 carbon atoms which may have a substituent group.)

A silacyclopentadiene derivative represented by the formula below is also preferable as a material of the electron injection (transport) layer.

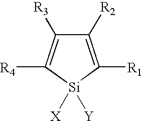

(In the formula, X and Y have a structure in which: X and Y each represent a saturated or unsaturated hydrocarbon group having 1 to 6 carbon atoms, an alkoxy group, an alkenyloxy group, an alkynyloxy group, a hydroxy group, a substituted or unsubstituted aryl group, a substituted or unsubstituted heterocycle; or X and Y are bonded to form a saturated or unsaturated ring. R₁ to R₄ have a structure in which: R₁ to R₄ each represent hydrogen, halogen, a substituted or unsubstituted alkyl group having 1 to 6 carbon atoms, an alkoxy group, an aryloxy group, a perfluoroalkyl group, a perfluoroalkoxy group, an amino group, an alkylcarbonyl group, an arylcarbonyl group, an alkoxycarbonyl group, an aryloxycarbonyl group, an azo group, an alkylcarbonyloxy group, an arylcarbonyloxy group, an alkoxycarbonyloxy group, an aryloxycarbonyloxy group, a sulfinyl group, a sulfonyl group, a sulfanyl group, a silyl group, a carbamoyl group, an aryl group, a heterocyclic group, an alkenyl group, an alkynyl group, a nitro group, a formyl group, a nitroso group, a formyloxy group, an isocyano group, a cyanate group, an isocyanate group, a thiocyanate group, an isothiocyanate group or cyano group; or an adjacent set of R₁ to R₄ are condensed to form a substituted or unsubstituted ring. A silacyclopentadiene derivative represented by the formula below is also preferable as a material of the electron injection (transport) layer.

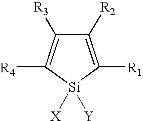

(In the formula, X and Y have a structure in which: X and Y each represent a saturated or unsaturated hydrocarbon group having 1 to 6 carbon atoms, an alkoxy group, an alkenyloxy group, an alkynyloxy group, a substituted or unsubstituted aryl group, a substituted or unsubstituted heterocycle; or X and Y are bonded to form a saturated or unsaturated ring. R₁ to R₄ have a structure in which: R₁ to R₄ each represent hydrogen, halogen, a substituted or unsubstituted alkyl group having 1 to 6 carbon atoms, an alkoxy group, an aryloxy group, a perfluoroalkyl group, a perfluoroalkoxy group, an amino group, an alkylcarbonyl group, an arylcarbonyl group, an alkoxycarbonyl group, an aryloxycarbonyl group, an azo group, an alkylcarbonyloxy group, an arylcarbonyloxy group, an alkoxycarbonyloxy group, an aryloxycarbonyloxy group, a sulfinyl group, a sulfonyl group, a sulfanyl group, a silyl group, a carbamoyl group, an aryl group, a heterocyclic group, an alkenyl group, an alkynyl group, a nitro group, a formyl group, a nitroso group, a formyloxy group, an isocyano group, a cyanate group, an isocyanate group, a thiocyanate group, an isothiocyanate group or cyano group; or an adjacent set of R₁ to R₄ are condensed to form a substituted or unsubstituted ring.

It should be noted that: when R₁ and R₄ are the phenyl group, X and Y are not the alkyl group and phenyl group; when R₁ and R₄ are a thienyl group, conditions of X and Y being a monovalent hydrocarbon group, R₂ and R₃ being the alkyl group, the aryl group or the alkenyl group and R₂ and R₃ being aliphatic groups bonded to form a ring are not satisfied at the same time; when R₁ and R₄ are the silyl group, R₂, R₃, X and Y each are not the monovalent hydrocarbon group having 1 to 6 carbon atoms or the hydrogen atom; and when benzene rings are condensed at R₁ and R₂, X and Y are not the alkyl group and the phenyl group.

A borane derivative represented by the formula below is also preferable as a material of the electron injection (transport) layer.

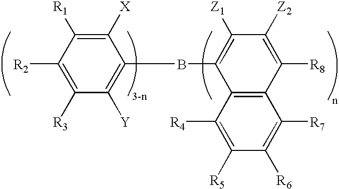

In the formula, R₁ to R₈ and Z₂ each represent a hydrogen atom, a saturated or unsaturated hydrocarbon group, an aromatic group, a heterocyclic group, a substituted amino group, a substituted boryl group, an alkoxy group or an aryloxy group; X, Y and Z₁ each represent a saturated or unsaturated hydrocarbon group, an aromatic group, a heterocyclic group, a substituted amino group, an alkoxy group or an aryloxy group; substituent groups of Z₁ and Z₂ may be bonded to form a condensed ring; and n represents an integer of 1 to 3, where when n is equal to or larger than 2, Z₁ may be different.

However a condition in which n is 1, X, Y and R₂ are the methyl group and R₈ is the hydrogen atom or the substituted boryl group and a condition in which n is 3 and Z₁ is the methyl group are excluded.)

A gallium complex represented by the formula below is also preferable as a material of the electron injection (transport) layer.

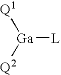

In this formula, Q¹ and Q² each represent a ligand shown by the formula below

L represents a ligand which may be a halogen atom; a substituted or unsubstituted alkyl group; a substituted or unsubstituted cycloalkyl group; a substituted or unsubstituted aryl group; a substituted or unsubstituted heterocyclic group; those represented by —OR¹ (R₁ representing a halogen atom, a substituted or unsubstituted alkyl group, a substituted or unsubstituted cycloalkyl group, a substituted or unsubstituted aryl group or a substituted or unsubstituted heterocyclic group); or those represented by —O-Ga-Q³(Q⁴) (Q³ and Q⁴ being the same as Q¹ and Q²).

In the formula, Q¹ to Q⁴ each represent a residue represented by the formula below, which may be exemplified by, but not limited to, a quinoline residue such as 8-hydroxyquinoline and 2-methyl-8-hydroxyquinoline.

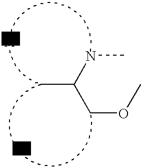

Rings A₁ and A₂ are bonded to each other, Rings A¹ and A² being substituted or unsubstituted aryl rings bonded to each other or a heterocyclic structure.

The metal complex shown above exhibits a strong property as an n-type semiconductor and has a large electron injection capability. In addition, formation energy required when forming the complex is low, so that bonding between the metal and the ligand in the formed metal complex becomes strong, thus exhibiting a large fluorescence quantum efficiency as a luminescent material.

Concrete examples of the substituent groups of Ring A¹ and Ring A² that form the ligands in the formula above may include: halogen atoms of chlorine, bromine, iodine and fluorine; substituted or unsubstituted alkyl groups such as a methyl group, an ethyl group, a propyl group, a butyl group, a sec-butyl group, a tert-butyl group, a pentyl group, a hexyl group, a heptyl group, an octyl group, a stearyl group and a trichloromethyl group; substituted or unsubstituted aryl groups such as a phenyl group, a naphthyl group a 3-methylphenyl group, a 3-methoxyphenyl group, a 3-fluorophenyl group, a 3-trichloromethylphenyl group, a 3-trifluoromethylphenyl group and a 3-nitrophenyl group; substituted or unsubstituted alkoxy groups such as a methoxy group, a n-butoxy group, a tert-butoxy group, a trichloromethoxy group, a trifluoroethoxy group, a pentafluoropropoxy group, a 2,2,3,3-tetrafluoropropoxy group, a 1,1,1,3,3,3-hexafluoro-2-propoxy group and a 6-(perfluorohexyl)hexyloxy group; substituted or unsubstituted aryloxy groups such as a phenoxy group, a p-nitrophenoxy group, a p-tert-butylphenoxy group, a 3-fluorophenoxy group, a pentafluorophenyl group and a 3-trifluoromethylphenoxy group; substituted or unsubstituted alkylthio groups such as a methylthio group, an ethylthio group, a tert-butylthio group, a hexylthio group, an octylthio group and a trifluoromethylthio group; substituted or unsubstituted arylthio groups such as a phenylthio group, a p-nitrophenylthio group, a p-tert-butylphenylthio group, a 3-fluorophenylthio group, a pentafluorophenylthio group and a 3-trifluoromethylphenylthio group; mono- or disubstituted amino groups such as a cyano group, a nitro group, an amino group, a methylamino group, a diethylamino group, an ethylamino group, a diethylamino group, a dipropylamino group, a dibutylamino group and a diphenylamino group; acylamino groups such as a bis(acetoxymethyl)amino group, a bis(acetoxyethyl)amino group, a bis (acetoxypropyl)amino group and a bis(acetoxybutyl)amino group; a hydroxyl group; a siloxy group; an acyl group; carbamoyl groups such as a methylcarbamoyl group, a dimethylcarbamoyl group, an ethylcarbamoyl group, a diethylcarbamoyl group, a propylcarbamoyl group, a butylcarbamoyl group, and a phenylcarbamoyl group; carboxylic acid groups; sulfonic acid groups; imide groups; cycloalkyl groups such as a cyclopentane group and a cyclohexyl group; aryl groups such as a phenyl group, a naphthyl group, a biphenyl group, an anthranil group, a phenanthryl group, a fluorenyl group and a pyrenyl group; and heterocyclic groups such as a pyridinyl group a pyrazinyl group, a pyrimidinyl group, a pyridazinyl group, a triazinyl group, an indolinyl group, a quinolinyl group, an acridinyl group, a pyrrolidinyl group, a dioxanyl group, a piperidinyl group, a morpholidinyl group, a piperazinyl group, a triathinyl group, a carbazolyl group, a furanyl group, a thiophenyl group, an oxazolyl group, an oxadiazolyl group, a benzoxazolyl group, a thiazolyl group, a thiadiazolyl group, a benzothiazolyl group, a triazolyl group, an imidazolyl group, a benzoimidazolyl group and a pranyl group. In addition, the substituent groups listed above may be bonded to each other to form a 6-membered aryl ring or a heterocycle.

As a preferred embodiment of the organic EL device, there is known a device containing a reductive dopant at a boundary between a region transporting the electron or the cathode and an organic layer. Here, the reductive dopant is defined as a substance capable of reducing an electron transporting compound. Thus, various substances having a certain level of reducibility can be used, preferable examples of which may be at least one substance selected from the group consisting of: alkali metal, alkali earth metal, rare earth metal, an oxide of the alkali metal, a halogenide of the alkali metal, an oxide of the alkali earth metal, a halogenide of the alkali earth metal, an oxide of the rare earth metal, a halogenide of the rare earth metal, an organic complex of the alkali metal, an organic complex of the alkali earth metal and an organic complex of the rare earth metal.

Specifically, more preferable reductive dopant may be those having the work function of 2.9 eV or lower, which may be exemplified by at least one alkali metal selected from the group consisting of Li (work function: 2.9 eV), Na (work function: 2.36 eV), K (work function: 2.28 eV), Rb (work function: 2.16 eV) and Cs (work function: 1.95 eV) or at least one alkali earth metal selected from the group consisting of Ca (work function: 2.9 eV), Sr (work function: 2.0 to 2.5 eV) and Ba (work function: 2.52 eV), and the substances having the work function of 2.9 eV or lower are particularly preferable. Among these, more preferable reductive dopant is at least one alkali metal selected from the group consisting of K, Rb and Cs, in which Rb and Cs are even more preferable and Cs is most preferable. These alkali metals have particularly high reducibility, so that addition of a relatively small amount of these alkali metals to an electron injection region can enhance luminescence intensity and lifecycle of the organic EL device. In addition, as the reductive dopant having the work function of 2.9 eV or lower, a combination of two or more of these alkali metals is also preferable, and a combination including Cs is particularly preferable, e.g., combinations of Cs an Na, Cs and K, Cs and Rb or Cs, Na and K. The combinations including Cs can effectively exert the reducibility, so that by adding such reductive dopant to the electron injection region, the luminescence intensity and the lifecycle of the organic EL device can be enhanced.

An electron injection layer formed from an insulator or a semiconductor may be provided between the cathode and the organic layer. With the arrangement, leak of electric current can be effectively prevented and the electron injection capability can be enhanced. For the semiconductor, it is preferable to use at least one metal compound selected from the group consisting of an alkali metal chalcogenide, an alkaline earth metal chalcogenide, a halogenide of alkali metal and a halogenide of alkali earth metal. By forming the electron injection layer from the alkali metal chalcogenide or the like, the electron injection capability can further be enhanced, which is preferable. Specifically, preferable examples of the alkali metal chalcogenide may include Li₂O, K₂O, Na₂S, Na₂Se and Na₂O, while preferable example of the alkaline earth metal chalcogenide may include CaO, BaO, SrO, BeO, BaS and CaSe. Preferable examples of the halogenide of the alkali metal may include LiF, NaF, KF, LiCl, KCl and NaCl. Preferable examples of the halogenide of the alkali earth metal may include fluorides such as CaF₂, BaF₂, SrF₂, MgF₂ and BeF₂ and halogenides other than the fluoride.

Examples of the semiconductor for forming the electron transport layer may include one type or a combination of two or more types of an oxide, a nitride or an oxidized nitride containing at least one element selected from the group consisting of Ba, Ca, Sr, Yb, Al, Ga, In, Li, Na, Cd, Mg, Si, Ta, Sb and Zn. An inorganic compound for forming the electron transport layer is preferably a microcrystalline or amorphous semiconductor film. When the electron transport layer is formed of such semiconductor film, more uniform thin film can be formed, thereby reducing pixel defects such as a dark spot. Examples of such inorganic compound may include the above-described alkali metal chalcogenide, alkali earth metal chalcogenide, halogenide of the alkali metal and halogenide of the alkali earth metal.

### (7) Cathode

In the cathode, metals, alloys, electrically conductive compounds and mixtures of the above, which each have a small work function (4 eV or lower), are used as an electrode material, in order to inject the electron to the electron injection/transport layer or the luminescent layer. Concrete examples of the electrode material may include sodium, a sodium-potassium alloy, magnesium, lithium, a magnesium-silver alloy, aluminium/aluminium oxide, an aluminium-lithium alloy, indium and rare earth metal.

The cathode may be made by forming a thin film from these electrode substances by the vapor deposition and sputtering.

When luminescence from the luminescent layer is taken out from the cathode, the cathode preferably has a transmittance of higher than 10% for the luminescence.

The sheet resistance as the cathode is preferably several hundreds Ω/square or lower, and the thickness of the film is typically in the range from 10 nm to 1 μm, preferably 50 to 200 nm.

### (8) Insulating Layer

Since the electrical field is applied to ultra thin films in the organic EL device, pixel defects resulted from leak or short circuit likely occur. In order to prevent such defects, it is preferable to interpose an insulating thin film layer between a pair of electrodes.

Examples of materials used for the insulating layer may include aluminum oxide, lithium fluoride, lithium oxide, cesium fluoride, cesium oxide, magnesium oxide, magnesium fluoride, calcium oxide, calcium fluoride, aluminium nitride, titanium oxide, silicon oxide, germanium oxide, silicon nitride, boron nitride, molybdenum oxide, ruthenium oxide and vanadium oxide.

Mixtures or laminates of the above may also be used.

### (9) Manufacturing Method of Organic EL Device

The organic EL device can be manufactured by forming the anode, the luminescent layer, the hole injection layer (as needed), the electron injection layer (as needed) and the cathode using the materials and formation methods mentioned above as examples. Also, the organic EL device can be manufactured by forming the above elements in the inverse order of the above, namely from the cathode to the anode.

The following is an example of a manufacturing method of the organic EL device in which the anode, the hole injection layer, the luminescent layer, the electron injection layer and the cathode are sequentially formed on the light-transmissive substrate.

First, a thin film of the anode material is formed on a suitable light-transmissive substrate by the vapor deposition or the sputtering such that the thickness of the thin film is 1 μm or smaller, preferably in the range from 10 nm to 200 nm. Then, the hole injection layer is formed on the anode.

The hole injection layer can be formed by the vacuum deposition, the spin coating, the casting method, the LB method or the like. The thickness of the hole injection layer is properly selected from the range from 5 nm to 5 μm.

Then, the luminescent layer is formed on the hole injection layer by forming a thin film from an organic luminescent material by a dry process represented by the vacuum deposition or a wet process such as the spin coating and the casting method. However, the wet process is more preferable in terms of size increase in screen, reduction of cost and simplification of manufacturing process.

Then, the electron injection layer is formed on the luminescent layer.

The vacuum deposition can be exemplified as a method for forming the electron injection layer.

Lastly, the cathode is deposited, and the organic EL device can be obtained.

The cathode is formed from metal by the vapor deposition, the sputtering or the like.

In order to protect the organic layers deposited under the cathode from being damaged, the vacuum deposition is preferable.

The methods for forming each of the layers of the organic EL device are not particularly limited.

Conventional methods such as the vacuum deposition and the spin coating can be employed for forming the organic film layers. Specifically, the organic film layers may be formed by conventional coating methods such as the vacuum deposition, molecular beam epitaxy (MBE method) and coating methods using a solution such as a dipping, the spin coating, the casting method, bar coating, roll coating and ink jet printing. Although the thickness of each organic layer of the organic EL device is not particularly limited, the thickness is generally preferably in the range from several nanometers to 1 μm, since too small thickness likely cause defects such as a pin hole while too large thickness requires high voltage to be applied and lowers efficiency.

In a state where a direct current is applied to the organic EL device, when a voltage of 5 to 40 V is applied with the anode having the positive polarity and the cathode having the negative polarity, the luminescence can be observed. When the voltage is applied with the inversed polarity, the current is not applied, so that the luminescence is not generated. In a state where an alternating current is applied, the uniform luminescence can be observed only when the anode has the positive polarity and the cathode has the negative polarity. A waveform of the alternating current to be applied can be selected arbitrarily.

### Example 47

As Example 47, the organic EL device was manufactured as described below.

A glass substrate (size: 25 mm×75 mm×1.1 mm thick) having an ITO transparent electrode (manufactured by GEOMATEC Co., Ltd.) is ultrasonic-cleaned in isopropyl alcohol for five minutes, and then UV/ozone-cleaned for 30 minutes.

Polyethylene-dioxy-thiophene/polystyrene sulphonic acid (PEDOT/PSS) to be used for the hole injection layer was deposited on the substrate by the spin coating to form a film having a thickness of 100 nm.

Then, a toluene solution (0.6 wt %) of Polymer 1 shown below (Mw: 145000) was deposited by the spin coating to form a film having a thickness 20 nm, which was dried at 170° C. for 30 minutes.

Next, the luminescent layer was formed by the spin coating using Ink 28 of the example described above. The thickness was 50 nm at this time.

Then, on the luminescent layer, a tris(8-quinolinol)aluminum film (hereinafter, abbreviated as Alq film) having a thickness of 10 nm was formed.

The Alq film serves as the electron transport layer. Li (Li source: manufactured by SAES Getters) as the reductive dopant and Alq are co-deposited to form an Alq:Li film as the electron injection layer (cathode).

Metal (Al) was vapor-deposited on the Alq:Li film to form a metal cathode to complete the organic EL device.

The device emitted a blue light and had a uniform light emission surface.

The luminescence efficiency at this time was 5.5 cd/A, and time elapsed until the luminescent intensity decreased to half was 1600 hours with the initial luminescence intensity being 1000 cd/m².

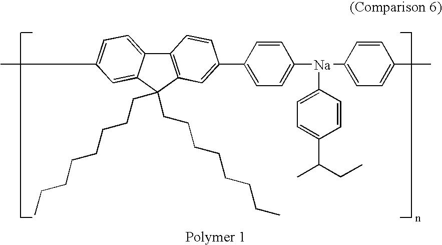

Compound H10 (solubility in toluene: 5 wt %) was used instead of host Compound H4 in Example 28. The ink had no solid matters left undissolved, and the precipitation was not observed after a week.

Although the device was manufactured using this ink and by the method same as Example 47, the luminescence efficiency was 4.1 cd/A, and time elapsed until the luminescent intensity decreased to half was 460 hours with the initial luminescence intensity being 1000 cd/m²

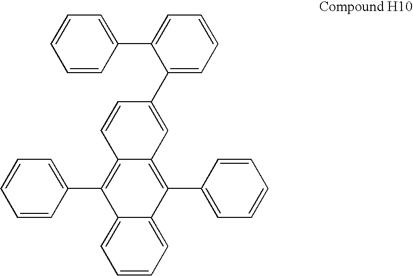

As obvious from the result above, providing the substituent in the position 2 of the anthracene can enhance the solubility in the solvent, but sacrifices the luminescence property of the device. That is to say, substituting the positions 9 and 10 of the anthracene by a specific structure is important in order to enhance both the solubility and the performance of the device, and the present invention provides the compounds achieving both of the aspects.

It should be noted that the present invention is not limited to the examples above or the like, but may be appropriately modified within the scope of the present invention.

The basic application Number JP2006-304627 upon which this patent application is based is hereby incorporated by reference.

1. An organic EL material-containing solution, comprising:
an organic EL material;
a solvent; and
a viscosity control agent, wherein
the organic EL material contains a host and a dopant,
the host is a compound shown by Formula (1) below, and
the host has a solubility of 2 wt % or higher in the solvent, 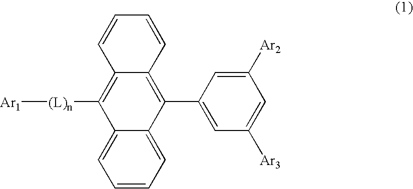
(Ar₁ to Ar₃ each representing a substituted or unsubstituted aryl group or heteroaryl group having 5 to 50 nuclear atoms or a condensed aromatic group having 10 to 30 carbon atoms, L representing a single bond or a divalent linking group, the divalent linking group being a substituted or unsubstituted arylene group or heteroarylene group having 5 to 50 nuclear atoms, n representing an integer of 1 to 4).

2. The organic EL material-containing solution according to claim 1, wherein Ar₁ to Ar₃ each are a substituted or unsubstituted aryl group having 5 to 50 nuclear atoms.

3. The organic EL material-containing solution according to claim 1, wherein Ar₁ to Ar₃ each are a substituted or unsubstituted phenyl group or a naphthyl group.

4. The organic EL material-containing solution according to claim 1, wherein the host is a compound shown by Formula (2) below instead of the compound shown above in Formula (1), 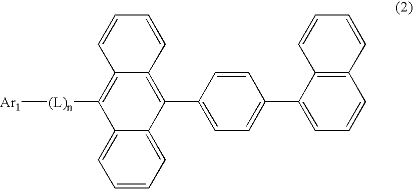
(Ar₁ representing a substituted or unsubstituted aryl group or heteroaryl group having 5 to 50 nuclear atoms, L representing a single bond or a divalent linking group, the divalent linking group being a substituted or unsubstituted arylene group or heteroarylene group having 5 to 50 nuclear atoms or a condensed aromatic group having 10 to 30 carbon atoms, n representing an integer of 1 to 4).

5. The organic EL material-containing solution according to claim 4, wherein Ar₁ is a substituted or unsubstituted aryl group having 5 to 50 nuclear atoms.

6. The organic EL material-containing solution according to claim 4, wherein Ar₁ is a substituted or unsubstituted phenyl group or a naphthyl group.

7. The organic EL material-containing solution according to claim 1, wherein n is 1 or 2.

8. The organic EL material-containing solution according to claim 1, wherein the dopant is a styrylamine derivative shown by Formula (3) below, the dopant having a substituent group that is a straight-chained or branched alkyl group having 2 to 6 carbon atoms or a cycloalkyl group having 5 to 10 carbon atoms, and the dopant material has a solubility of 0.5 wt % or higher in the solvent, 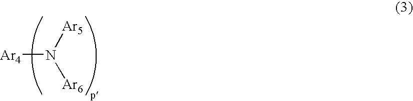
(at least one of Ar₄ to Ar₆ containing a styryl group).

9. The organic EL material-containing solution according to claim 8, wherein the dopant material is, instead of the styrylamine derivative shown by Formula (3), a substituted derivative of an arylamine shown by Formula (4) below, the substituted derivative of the arylamine having as a substituent group a straight-chained or branched alkyl group having 2 to 6 carbon atoms or a cycloalkyl group having 5 to 10 carbon atoms, 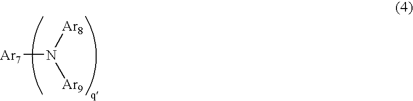
(Ar₇ to Ar₉ each representing a substituted or unsubstituted aryl group having 5 to 40 nuclear carbon atoms, q′ representing an integer of 1 to 4).

10. The organic EL material-containing solution according to claim 1, wherein
the solvent is selected from the group consisting of an aromatic solvent, a halogen type solvent and an ether type solvent, and
the viscosity control agent is selected from the group consisting of an alcohol type solution, a ketone type solution, a paraffin type solution and an alkyl-substituted aromatic solution having 4 or more carbon atoms.

11. The organic EL material-containing solution according to claim 1, wherein
the solvent is the aromatic solvent, and
the viscosity control agent is the alcohol type solution or the alkyl-substituted aromatic solution having 4 or more carbon atoms.

12. An organic EL material-film forming method, comprising:
a dropping step for dropping an organic EL material-containing solution in a film formation area, the organic EL material-containing solution containing an organic EL material, a solvent and a viscosity control agent; and
a film forming step for evaporating the solvent in the organic EL material-containing solution dropped in the dropping step to form a thin film of the organic EL material, wherein
the organic EL material contains a host and a dopant,
the host is a compound shown by Formula (1) below, and
the host has a solubility of 2 wt % or higher in the solvent, 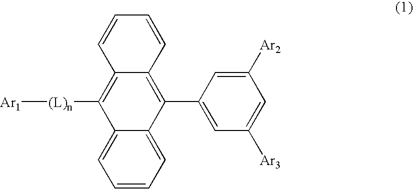
(Ar₁ to Ar₃ each representing a substituted or unsubstituted aryl group or heteroaryl group having 5 to 50 nuclear atoms or a condensed aromatic group having 10 to 30 carbon atoms, L representing a single bond or a divalent linking group, the divalent linking group being a substituted or unsubstituted arylene group or heteroarylene group having 5 to 50 nuclear atoms, n representing an integer of 1 to 4).

13. A thin film of an organic EL material that is formed by an organic EL material-film forming method, wherein
the organic EL material-film forming method includes:
a dropping step for dropping an organic EL material-containing solution in a film formation area, the organic EL material-containing solution containing an organic EL material, a solvent and a viscosity control agent; and
a film forming step for evaporating the solvent in the organic EL material-containing solution dropped in the dropping step to form a thin film of the organic EL material,
the organic EL material contains a host and a dopant,
the host is a compound shown by Formula (1) below, and
the host has a solubility of 2 wt % or higher in the solvent, 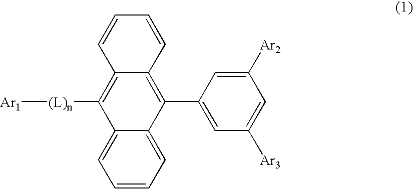
(Ar₁ to Ar₃ each representing a substituted or unsubstituted aryl group or heteroaryl group having 5 to 50 nuclear atoms or a condensed aromatic group having 10 to 30 carbon atoms, L representing a single bond or a divalent linking group, the divalent linking group being a substituted or unsubstituted arylene group or heteroarylene group having 5 to 50 nuclear atoms, n representing an integer of 1 to 4).

14. An organic EL device, comprising: a thin film of an organic EL material, wherein
the thin film of the organic EL material is formed by an organic EL material-film forming method,
the organic EL material-film forming method includes:
a dropping step for dropping an organic EL material-containing solution in a film formation area, the organic EL material-containing solution containing an organic EL material, a solvent and a viscosity control agent; and
a film forming step for evaporating the solvent in the organic EL material-containing solution dropped in the dropping step to form a thin film of the organic EL material,
the organic EL material contains a host and a dopant,
the host is a compound shown by Formula (1) below, and
the host has a solubility of 2 wt % or higher in the solvent, 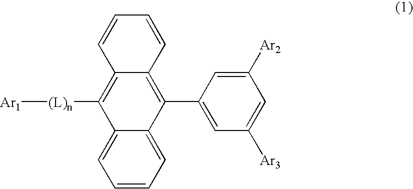
(Ar₁ to Ar₃ each representing a substituted or unsubstituted aryl group or heteroaryl group having 5 to 50 nuclear atoms or a condensed aromatic group having 10 to 30 carbon atoms, L representing a single bond or a divalent linking group, the divalent linking group being a substituted or unsubstituted arylene group or heteroarylene group having 5 to 50 nuclear atoms, n representing an integer of 1 to 4).

15. The organic EL material-containing solution according to claim 4, wherein n is 1 or 2.

16. The organic EL material-containing solution according to claim 4, wherein
the dopant is a styrylamine derivative shown by Formula (3) below, the dopant having a substituent group that is a straight-chained or branched alkyl group having 2 to 6 carbon atoms or a cycloalkyl group having 5 to 10 carbon atoms, and
the dopant has a solubility of 0.5 wt % or higher in the solvent, 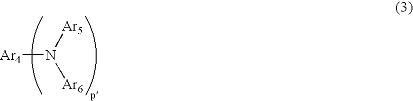
(at least one of Ar₄ to Ar₆ containing a styryl group).

17. The organic EL material-containing solution according to claim 16, wherein the dopant is, instead of the styrylamine derivative shown by Formula (3), a substituted derivative of an arylamine shown by Formula (4) below, the substituted derivative of the arylamine having as a substituent group a straight-chained or branched alkyl group having 2 to 6 carbon atoms or a cycloalkyl group having 5 to 10 carbon atoms, 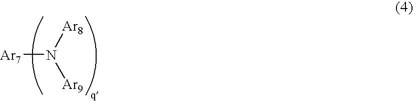
(Ar₇ to Ar₉ representing a substituted or unsubstituted aryl group having 5 to 40 nuclear carbon atoms, q′ representing an integer of 1 to 4).

18. The organic EL material-containing solution according to claim 4, wherein
the solvent is selected from the group consisting of an aromatic solvent, a halogen type solvent and an ether type solvent, and
the viscosity control agent is selected from the group consisting of an alcohol type solution, a ketone type solution, a paraffin type solution and an alkyl-substituted aromatic solution having 4 or more carbon atoms.

19. The organic EL material-containing solution according to claim 4, wherein
the solvent is the aromatic solvent, and
the viscosity control agent is the alcohol type solution or the alkyl-substituted aromatic solution having 4 or more carbon atoms.

20. The organic EL material-film forming method according to claim 12, wherein the organic EL material contains as a host a compound shown by Formula (2) below instead of the compound shown above in Formula (1), 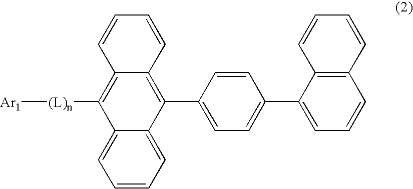
(Ar₁ representing the substituted or unsubstituted aryl group or heteroaryl group having 5 to 50 nuclear atoms, L representing a single bond or a divalent linking group, the divalent linking group being a substituted or unsubstituted arylene group or heteroarylene group having 5 to 50 nuclear atoms or a condensed aromatic group having 10 to 30 carbon atoms, n representing an integer of 1 to 4).

21. The thin film of the organic EL material according to claim 13, wherein the organic EL material contains as a host a compound shown by Formula (2) below instead of the compound shown above by Formula (1), 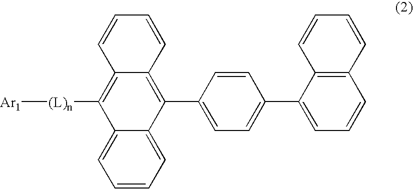
(Ar₁ representing a substituted or unsubstituted aryl group or heteroaryl group having 5 to 50 nuclear atoms, L representing a single bond or a divalent linking group, the divalent linking group being a substituted or unsubstituted arylene group or heteroarylene group having 5 to 50 nuclear atoms or a condensed aromatic group having 10 to 30 carbon atoms, n representing an integer of 1 to 4).

22. The organic EL device according to claim 14, wherein the organic EL material contains as a host a compound shown by Formula (2) below instead of the compound shown above in Formula (1), 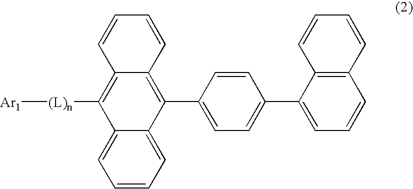
(Ar₁ representing a substituted or unsubstituted aryl group or heteroaryl group having 5 to 50 nuclear atoms, L representing a single bond or a divalent linking group, the divalent linking group being a substituted or unsubstituted arylene group or heteroarylene group having 5 to 50 nuclear atoms or a condensed aromatic group having 10 to 30 carbon atoms, n representing an integer of 1 to 4).
